(19) United States
(12) Patent Application Publication
(10) Pub. No.: US 2010/0122379 A1
(43) Pub. Date: May 13, 2010

(54) Active Ingredient Compositions for Plant Protection

(75) Inventors: Yvonne Dieckmann, Hassloch (DE); Mario Brands, Ludwigshafen (DE); Rafel Israels, Koeln (DE); Thomas Friedrich, Darmstadt (DE); Gabi Winter, Wachenheim (DE); Motonori Yamamoto, Mannheim (DE); Peter Dombo, Wesbaden (DE); Ursula Huber-Moulliet, Frankenthal (DE); Silvia Sztoj, Ludwigshafen (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/597,113

(22) PCT Filed: Apr. 25, 2008
(86) PCT No.: PCT/EP08/55101

§ 371 Date: Oct. 22, 2009

(30) Foreign Application Priority Data
Apr. 26, 2007 (EP) 07107015.5
Apr. 26, 2007 (EP) 07107054.4

(51) Int. Cl. A01N 63/00; A01N 25/00; A01N 25/12; A01H 5/00

(52) U.S. Cl. 800/295; 424/499; 424/94.61; 424/94.63; 424/94.6

(57) ABSTRACT

The present invention relates to novel active substance compositions for plant protection in the form of finely divided active-substance-containing particles which compositions comprise
a) at least one plant protectant;
b) at least one polymer P which is not soluble in water and which is degradable by enzymatic hydrolysis, in an amount of at least 20% by weight based on the total amount of the components of the active-substance-comprising particles, and
c) at least one hydrolase (EC 3),
   where at least 90% by weight of the active substance-containing particles of the active substance composition do not exceed a diameter of 500 μm (D₉₀-value) and where components a), b) and c) account for at least 30% by weight of the active substance particles.

The invention also relates to a process for their preparation, and to the use of the active substance compositions for the treatment of plants, soils and of seed.

The present invention relates to novel active substance compositions for plant protection in the form of finely particulate, active substance-containing particles, to a process for their preparation, and to the use of the active substance compositions for the treatment of plants, soils and of seed.

The protection of useful plants or crop plants from attack by plant-injurious organisms, the controlled regulation of the growth of useful plants, but also combating harmful plants by the application of suitable plant protection products are important instruments of increasing yields and thus also of safeguarding the production of plant-based foods.

The application of conventional plant protection products which serve for combating harmful organisms frequently entails disadvantages. Thus, the organism to be combated may develop resistances, in particular when the application is effected over a prolonged period, or not competently. Moreover, the long-term or incompetent application of plant protection products may lead to environmental problems or harm the user. This is why attempts are being made to find use forms for plant protection products which make possible a controlled application and thus a reduced application rate of plant protection products.

Many plants, in particular crop plants, are highly sensitive in the phase before and during germination and sprouting to attack by phytopathogenic fungi or animal pests. This can be attributed firstly to the small size of the plant parts, which makes it difficult for the plant to compensate for damage. On the other hand, the natural defense mechanisms of the plant are frequently not yet developed at this early growth stage. The protection of the plant before and during germination is therefore an important means for reducing plant damage.

At this early growth stage of the plant, the conventional application of plant protectants for combating plant-injurious organisms is frequently unsuccessful. Firstly, higher application rates may damage the plant itself. However, lower application rates usually do not permit an effective control of the harmful organisms. Conventional seed treatment is, in some respects, a way out. Here, the seed is treated, before or during planting, with a suitable active substance which is intended to protect the plant before or during germination or sprouting from attack by harmful organisms. A problem in conventional seed treatment is that planting the seed, and the germination and sprouting phase, are frequently some time apart. Within this period, the active substance can be leached by environmental factors, for example by rain, so that low application rates no longer guarantee sufficient protection. Higher application rates, in turn, lead to the abovementioned problems and additionally constitute a not inconsiderable expenditure. Other problems in conventional seed treatment are phytotoxic side effects and negative effects on the plants' growth by the active substance(s) applied to the seed. Again, a controlled, i.e. delayed, release of active substance should remedy this or at least reduce these problems.

There has therefore been no lack of attempts to formulate active substances in a manner which permits a controlled release of the active substance.

Thus, WO 99/00013 describes an active substance composition for the controlled release of a plant protectant, which composition consists of finely particulate active substance particles, the plant protectant being distributed in a polymeric matrix. As a rule, the polymeric matrix consists of one polymer which is not soluble in water and one polymer which is. Typically, the active substance is released as the result of decomposition of the active substance-containing particles, which is initiated by the water-soluble polymer constituent of the particles being dissolved. While leaching of the active substance can be diminished in this manner and a more uniform release of active substance achieved, the release of the active substance depends greatly on the moisture conditions in the soil.

JP 2002/360665, in turn, discloses macroscopic capsules with dimensions within the range of several millimeters, which capsules have a core and at least two coatings which enclose the core. The outer coating has at least one enzyme by means of which the outer layer or the layer lying underneath is degraded. These capsules can be employed as artificial seed or for administering medicaments. Such capsules are not suitable for the treatment of soil or of seed.

Thus, the problem on which the present invention is based is to provide active substance compositions which are suitable for the treatment of seed or of the soil and which permit a controlled release of the active substance, which does not depend on the moisture content of the soil.

Surprisingly, this problem was solved by the active substance compositions described hereinbelow.

The present invention thus relates to an active substance composition in the form of finely divided, active substance-containing particles, which composition comprises
a) at least one plant protectant;
b) at least one polymer P which is not soluble in water and which is degradable by enzymatic hydrolysis, in an amount of at least 20% by weight, frequently at least 30% by weight, preferably at least 40% by weight, in particular at least 45% by weight and especially at least 50% by weight, based on the total amount of the components of the active substance-containing particles, and
c) at least one hydrolase (EC 3),
   where at least 90% by weight of the active substance-containing particles of the active substance composition do not exceed a diameter of 500 μm (D₉₀-value) and where components a), b) and c) account for at least 30% by weight, frequently at least 40% by weight, in particular at least 50% by weight, especially at least 60% by weight, of the active substance particles.

In the active compound compositions according to the invention, the hydrolase brings about an enzymatic degradation of the polymer P, which leads to disintegration of the active substance particles and thus to a release of the active substance from the particles. Unlike in the finely-divided, polymer-encapsulated active substance compositions of the prior art, the active substance particles disintegrate not as the result of moisture alone, but additionally require a sufficient temperature since otherwise the activity of the hydrolase is too low to bring about an efficient degradation of the polymer P. However, a sufficient activity of the hydrolase is, as a rule, present when the temperatures reach ranges where the plants grow or seed starts to germinate or to sprout. As a result the compositions according to the invention ensure that the active substance is released at the point in time at which it is required by the plant. Thus, the active substance compositions according to the invention are particularly suitable for the treatment of seed and of the soil.

Accordingly, the present invention furthermore relates to the use of the active substance compositions for the treatment of the soil and for the treatment of seed. The present invention furthermore relates to seed which comprises such an active substance composition.

The active substance composition according to the invention also leads to better tolerance and increased efficiency in the treatment of plants. The present invention therefore also relates to the use of the active substance compositions for the treatment of plants.

The active substance compositions according to the invention comprise at least one polymer P which is degradable by enzymatic hydrolysis, but which itself is not soluble in water. A polymer which is not soluble in water is understood as meaning those polymers where a sample of 5 g in 1 liter of water at 25° C. has not dissolved completely even after a period of 48 hours has elapsed.

Polymers which can be degraded by enzymatic hydrolysis are, as a rule, those polymers which are known to the skilled worker as biodegradable polymers, i.e. polymers which comply with the definition of biodegradability as specified in DIN V 54900.

In general, biodegradability means that the polymers disintegrate within an appropriate and detectable period of time. As a rule, degradation takes place hydrolytically; it is predominantly caused by the effect of microorganisms such as bacteria, yeasts, fungi and algae, or by the hydrolases comprised therein. Biodegradability can be determined for example by mixing polymers with compost and storing the mixture for a specific period of time. In accordance with ASTM D 5338, ASTM D 6400 and DIN V 54900, CO₂-free air is allowed to pass for example through mature compost during the composting process, and this compost is subjected to a defined temperature regime. This defines the biodegradability via the ratio of the net CO₂ liberation of the sample (after subtracting the CO₂ liberation by the compost without sample) to the maximal CO₂ liberation of the sample (calculated via the carbon content of the sample). As a rule, biodegradable polymers, in particular biodegradable polyesters demonstrate pronounced degradation symptoms such as fungal colonization, tearing and pore formation after only a few days' composting. Such polymers are known to the skilled worker and commercially available.

Polymers P which are degradable by enzymatic hydrolysis have, as a rule, a multiplicity of hydrolyzable functional groups in the polymer backbone. As a rule, they take the form of ester or amide groups, urea groups, urethane groups or acetal groups. As an alternative, a polymer which is degradable by enzymatic hydrolysis may also have hydrolyzable functional groups which are bound to the polymer backbone and which impart an increased water solubility to the polymer after the hydrolysis.

The molecular weight of the polymers P which are degradable by enzymatic hydrolysis may be varied over wide ranges for the composition according to the invention and is typically in the range of from 1000 to 1 000 000, frequently in the range of from 5000 to 500 000 and specifically in the range of from 10 000 to 2 500 000 (number average).

Preferably, the polymers P have a melting point or a glass transition temperature of above 40° C., for example in the range of from 40 to 180° C. and in particular in the range of from 60 to 160° C.

Preferably the polymer P has a multiplicity of hydrolyzable functional groups, in particular ester groups, in the polymer backbone.

The polymers with a multiplicity of ester groups in the polymer backbone are understood to mean in particular polylactides, polycaprolactone, block copolymers of polylactide and poly-C₂-C₄-alkylene glycol, block copolymers of polycaprolactone and poly-C₂-C₄-alkylene glycol and the copolyesters defined hereinbelow, which are composed of at least aliphatic or cycloaliphatic dicarboxylic acid or an ester-forming derivative thereof and at least one aliphatic or cycloaliphatic diol component and, if appropriate, further components.

The term “polylactides” is understood as meaning polycondensates of lactic acid. Suitable polylactides are described in WO 97/41836, WO 96/18591, WO 94/05484,U.S. Pat. No. 5,310,865, U.S. Pat. No. 5,428,126, U.S. Pat. No. 5,440,008, U.S. Pat. No. 5,142,023, U.S. Pat. No. 5,247,058, U.S. Pat. No. 5,247,059, U.S. Pat. No. 5,484,881, WO 98/09613, U.S. Pat. No. 4,045,418, U.S. Pat. No. 4,057,537 and in Adv. Mater. 2000, 12, 1841-1846. These products take the form of polymers which are based on lactidic acid lactone (A), which is converted via ring-opening polymerization into polylactidic acid polymers (B):

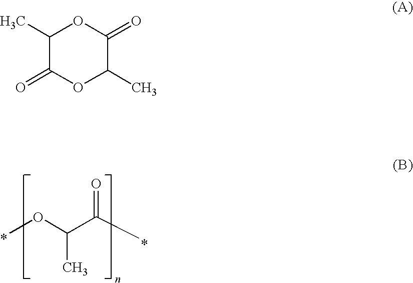

In formula (B), the degree of polymerization n is in the range of from 1000 to 4000, preferably from 1500 to 3500 and especially preferably from 1500 to 2000 (number average). The average molar masses (number average) of these products are, depending on the degree of polymerization, in the range of from 71,000 to 284,000 g/mol. Suitable polylactides can be obtained for example from Cargill Dow LLC (for example PLA polymer 4041 D, PLA polymer 4040D, PLA polymer 4031 D, PLA polymer 2000D or PLA polymer 1100) or Mitsui Chemicals (Lactea). Especially preferred polymers of the formula (B) have average molar masses (number average) of from 118,000 g/mol (Lactea), 212,000 g/mol (PLA polymer 4041D), and 223,000 g/mol (PLA polymer 2000D), respectively.

Also suitable are diblock and triblock copolymers of polylactides and poly-C₂-C₄-alkylene glycol, in particular with poly(ethylene glycol). These block copolymers are available for example from Aldrich (for example product number 659649). They may take the form of polymers which have polylactide blocks and poly-C₂-C₄-alkylene oxide blocks. Such block copolymers can be obtained for example by condensation of lactic acid or by ring-opening polymerization of lactidic acid lactone (A) in the presence of poly-C₂-C₄-alkylene glycols.

Polymers P which are suitable in accordance with the invention are, in particular, polycaprolactones. These are understood by the skilled worker to mean polymers which are described by formula D hereinbelow, where n is the number of recurring units in the polymer, i.e. the degree of polymerization.

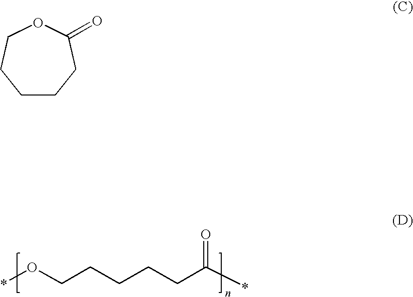

In formula (D), the degree of polymerization n is in the range of from 100 to 1000, preferably 500 to 1000 (number average). The number-average molecular weights of these products are, depending on the degree of polymerization, in the range of from 10 000 g/mol to 100 000 g/mol. Especially preferred polymers of the formula (D) have average molar masses (number average) of 50 000 g/mol (CAPA 6500), 80 000 g/mol (CAPA 6800) and 100 000 g/mol (CAPA FB 100).

As a rule, polycaprolactones are prepared by ring-opening polymerization of ε-caprolactone (compound C) in the presence of a catalyst.

Polycaprolactones are commercially available from Solvay under the name CAPA polymers, for example CAPA 6100, 6250, 6500 or CAPA FB 100.

Suitable polymers P are furthermore diblock and triblock copolymers of polycaprolactone with poly-C₂-C₄-alkylene glycols, in particular with polyethylene glycols (=polyethylene oxides), i.e. polymers which have at least one polycaprolactone block of the formula D and at least one polyalkylene glycol block. Such polymers can be prepared for example by polymerization of caprolactone in the presence of polyalkylene glycols, for example analogously to the processes described in Macromolecules 2003, 36, pp 8825-8829.

Other polymers P which are suitable in accordance with the invention are in particular copolyesters which are composed of at least one aliphatic or cycloaliphatic dicarboxylic acid or one ester-forming derivative thereof and at least one aliphatic or cycloaliphatic diol component and, if appropriate, further components. In particular, they take the form of copolyesters which are composed of:
A) an acid component which comprises
   a1) 30 to 100 mol % of at least one aliphatic or at least one cycloaliphatic dicarboxylic acid or ester-forming derivatives or mixtures thereof,
   a2) 0 to 70 mol % of at least one aromatic dicarboxylic acid or their ester-forming derivative or mixtures thereof and
   a3) 0 to 5 mol % of a sulfonate-containing compound with at least two carboxyl groups,
   where the molar percentages of components a1) to a3) together add up to 100% and,
B) a diol component selected among C₂-C₁₂-alkane diols, C₅-C₁₀-cycloalkane diols and mixtures of these,
   and, if desired,
C) one or more esterifiable components which are other than A and B, as components C.

Dicarboxylic acids a1) which are suitable in accordance with the invention generally have 2 to 10 carbon atoms, preferably 4 to 8 and in particular 6 carbon atoms. They may be both linear and branched. As a rule, the cycloaliphatic dicarboxylic acids which can be employed for the purposes of the present invention are those with 7 to 10 carbon atoms and in particular those with 8 carbon atoms. In principle, however, it is also possible to employ dicarboxylic acids with a larger number of carbon atoms, for example with up to 30 carbon atoms. Examples which may be mentioned are: malonic acid, succinic acid, glutaric acid, 2-methylglutaric acid, 3-methylglutaric acid, adipic acid, pimelic acid, acelaic acid, sebacic acid, fumaric acid, 2,2-dimethylglutaric acid, suberic acid, 1,3-cyclopentanedicarboxylic acid, 1,4-cyclohexanedicarboxylic acid, 1,3-cyclohexanedicarboxylic acid, diglycolic acid, itaconic acid, maleic acid and 2,5-norbornanedicarboxylic acid. Ester-forming derivatives of the above-mentioned aliphatic or cycloaliphatic dicarboxylic acids which can likewise be employed are, in particular, the di-C₁-to-C₆-alkyl esters, such as dimethyl, diethyl, di-n-propyl, diisopropyl, di-n-butyl, diisobutyl, di-t-butyl, di-n-pentyl, diisopentyl or di-n-hexyl esters. Anhydrides of dicarboxylic acids can likewise be employed. Substances which are especially preferably employed are adipic acid or succinic acid, their respective ester-forming derivatives or mixtures thereof.

Aromatic dicarboxylic acids a2 which may be mentioned are generally those with 8 to 12 carbon atoms and preferably those with 8 carbon atoms. Examples which may be mentioned are terephthalic acid, isophthalic acid, 2,6-naphthoic acid and 1,5-naphthoic acid, and ester-forming derivatives thereof. The di-C₁-C₆-alkyl esters, for example dimethyl, diethyl, diethyl, di-n-propyl, diisopropyl, di-n-butyl, diisobutyl, di-t-butyl, di-n-pentyl, diisopentyl or di-n-hexyl esters may be mentioned in particular in this context. The anhydrides of the dicarboxylic acids a2 are also suitable ester-forming derivatives. In principle, however, it is also possible to employ aromatic dicarboxylic acids a2 with a larger number of carbon atoms, for example up to 20 carbon atoms. The aromatic dicarboxylic acids or their ester-forming derivatives a2 can be employed individually or as a mixture of two or more of these. Terephthalic acid or its ester-forming derivatives, such as dimethyl terephthalate, are especially preferably used.

A sulfonate-containing compound which usually is employed is an alkali or alkaline earth metal salt of a sulfonate-containing dicarboxylic acid or its ester-forming derivatives, preferably alkali metal salts of 5-sulfoisophthalic acid or their mixtures, especially preferably the sodium salt.

In general, the diols B are selected among branched or linear alkane diols with 2 to 12 carbon atoms, preferably 4 to 8 or in particular 6 carbon atoms, or cycloalkane diols with 5 to 10 carbon atoms.

Examples of suitable alkane diols are ethylene glycol, 1,2-propanediol, 1,3-propane-diol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, in particular ethylene glycol, 1,3-propanediol, 1,4-butanediol and 2,2-dimethyl-1,3-propanediol (neopentyl glycol); cyclopentanediol, 1,4-cyclohexanediol, 1,2-cyclohexanedimethanol, 1,3-cyclohexane-dimethanol, 1,4-cyclohexanedimethanol or 2,2,4,4-tetramethyl-1,3-cyclobutanediol. Mixtures of different alkane diols may also be used.

Depending on whether an excess of acid or OH end groups is desired, either component A or component B may be employed in excess. In accordance with a preferred embodiment, the molar ratio of components A and B employed can be in the range of from 0.4:1 to 1.5:1, preferably in the range of from 0.6:1 to 1.1:1.

In addition to component A and B, the polyesters may comprise further components C and/or D incorporated. Components C include:
c1) dihydroxy compounds of the formula I

HO-[(A)-O]ₘ—H (I)
where A is a C₂-C₄-alkylene unit such as 1,2-ethanediyl, 1,2-propanediyl, 1,3-propanediyl or 1,4-butanediyl, and m is an integer from 2 to 250;
c2) hydroxycarboxylic acids of the formula IIa or IIb

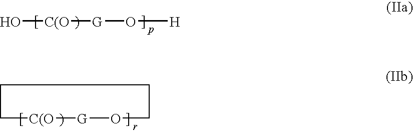

where p represents an integer from 1 to 1500 and r an integer from 1 to 4, and G is a radical which is selected from the group consisting of phenylene, —(CH₂)_{q}—, where q is an integer from 1 to 5, —C(R)H— and —C(R)HCH₂, where R is methyl or ethyl;
c3) amino-C₂-C₁₂-alkanols, amino-C₅-C₁₀-cycloalkanols or mixtures of these;
c4) diamino-C₁-C₈-alkanes;
c5) 2,2′-bisoxazolines of the general formula III

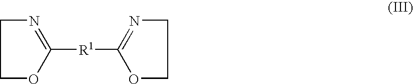

where R₁ represents a single bond, a (CH₂)_{z}-alkylene group, where z=2, 3 or 4, or a phenylene group;
c6) aminocarboxylic acids, selected among natural amino acids, polyamides with a molecular weight of not more than 18 000 g/mol, obtainable by polycondensation of a dicarboxylic acid with 4 to 6 C atoms and a diamine with 4 to 10 C atoms, compounds of the formulae IVa and IVb

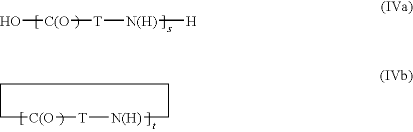

where s is an integer from 1 to 1500 and t is an integer from 1 to 4, and T is a radical which is selected from the group consisting of phenylene, —(CH₂)ᵤ—, where u is an integer from 1 to 12, —C(R²)H— and —C(R²)HCH₂, where R² is methyl or ethyl,
and polyoxazolines with the recurring unit V

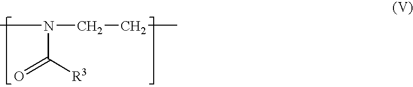

in which R³ represents hydrogen, C₁-C₆-alkyl, C₅-C₈-cycloalkyl, unsubstituted phenyl or phenyl which is up to trisubstituted by C₁-C₄-alkyl groups, or represents tetrahydrofuryl;
c7) compounds with at least three esterifiable groups;
c8) isocyanates and
c9) divinyl ethers.

Examples of component c1 are diethylene glycol, triethylene glycol, polyethylene glycol, polypropylene glycol and polytetrahydrofuran (poly-THF), especially preferably diethylene glycol, triethylene glycol and polyethylene glycol, it also being possible to employ mixtures of these or compounds which have different alkylene units A (see formula I), for example polyethylene glycol which comprises propylene units (A=1,2- or 1,3-propanediyl). The latter are for example obtainable by polymerization by methods known per se of first ethylene oxide and subsequently propylene oxide. Copolymers based on polyalkylene glycols, with different variables A, where units formed by ethylene oxide (A=1,2-ethanediyl) predominate, are particularly preferred. The molecular weight (number average Mₙ) of the polyethylene glycol is, as a rule, selected within the range of from 250 to 8000, preferably from 600 to 3000 g/mol.

In accordance with one of the preferred embodiments, it is possible to use, for the preparation of the copolyesters, for example 15 to 98, preferably 60 to 99.5 mol % of diols B and 0.2 to 85, preferably 0.5 to 30 mol % of the dihydroxy compounds c1, based on the molar amount of B and c1.

Examples of preferred components c2 are glycolic acid, D-, L-, D,L-lactic acid, 6-hydroxyhexanoic acid, its cyclic derivatives such as glycolide (1,4-dioxane-2,5-dione), D-, L-dilactide (3,6-dimethyl-1,4-dioxane-2,5-dione), p-hydroxybenzoic acid and its oligomers and polymers such as 3-polyhydroxybutyric acid, polyhydroxyvaleric acid, polylactide (for example obtainable under the name EcoPLA® (Cargill)) and a mixture of 3-polyhydroxybutyric acid and polyhydroxyvaleric acid (the latter being available from Zeneca under the name Biopol®). Especially preferred for the preparation of copolyesters are the low-molecular-weight and cyclic derivatives of these. The hydroxycarboxylic acids, or their oligomers and/or polymers, can be employed for example in amounts of from 0.01 to 50, preferably 0.1 to 40% by weight, based on the amounts of A and B.

Preferred components c3 are amino-C₂-C₆-alkanols such as 2-aminoethanol, 3-aminopropanol, 4-aminobutanol, 5-aminopentanol, 6-aminohexanol and amino-C₅-C₆-cycloalkanols such as aminocyclopentanol and aminocyclohexanol, or mixtures of these.

Preferred components c4) are diamino-C₄-C₆-alkanes such as 1,4-diaminobutane, 1,5-diaminopentane and 1,6-diaminohexane.

In accordance with a preferred embodiment, 0.5 to 99.5 mol %, preferably 0.5 to 50 mol %, of c3, based on the molar amount of B, and 0 to 50, preferably 0 to 35 mol %, of c4, based on the molar amount of B, are employed for the preparation of the copolyesters.

Preferred bisoxazolines III of component c5) are those in which R¹ denotes a single bond, a (CH₂)_{z}-alkylene group where z=2, 3 or 4, such as methylene, ethane-1,2-diyl, propane-1,3-diyl, propane-1,2-diyl, or a phenylene group. Those which may be mentioned as especially preferred bisoxazolines are 2,2′-bis(2-oxazoline), bis(2-oxazolinyl)methane, 1,2-bis(2-oxazolinyl)ethane, 1,3-bis(2-oxazolinyl)propane or 1,4-bis(2-oxazolinyl)butane, 1,4-bis(2-oxazolinyl)benzene, 1,2-bis(2-oxazolinyl)benzene or 1,3-bis(2-oxazolinyl)benzene. Bisoxazolines of the general formula III are generally obtainable by the method of Angew. Chem. Int. Edit., Vol. 11 (1972), p. 287-288.

To prepare the polyesters, it is possible to use, for example, from 70 to 98 mol % of B, up to 30 mol % of c3, up to 30 mol %, for example from 0.5 to 30 mol % of c4, and up to 30 mol %, for example from 0.5 to 30 mol % of c5, in each case based on the total of the molar amounts of components B, c3, c4 and c5. In accordance with another preferred embodiment, it is possible to employ from 0.1 to 5% by weight, preferably 0.2 to 4% by weight of c5, based on the total weight of A and B.

Natural aminocarboxylic acids may be employed as component c6. These include valine, leucine, isoleucine, threonine, methionine, phenylalanine, tryptophan, lysine, alanine, arginine, aspartic acid, cysteine, glutamic acid, glycine, histidine, proline, serine, tryosine, asparagine or glutamine.

Preferred aminocarboxylic acids of the general formulae IVa and IVb are those in which s is an integer from 1 to 1000 and t is an integer from 1 to 4, preferably 1 or 2, and T is selected from the group consisting of phenylene and —(CH₂)ᵤ—, where u is 1, 5 or 12.

Furthermore, c6 may also be a polyoxazoline of the general formula V. However, component c6 may also be a mixture of different aminocarboxylic acids and/or polyoxazolines.

In accordance with a preferred embodiment, c6 may be employed in amounts of from 0.01 to 50, preferably from 0.1 to 40, % by weight, based on the total amount of components A and B.

Further components which may optionally be employed to prepare the polyesters include compounds c7, which comprise at least three esterifiable groups. Compounds c7 comprise preferably three to ten functional groups which are capable of forming ester bonds. Especially preferred compounds c7 have three to six functional groups of this type in the molecule, in particular three to six hydroxyl groups and/or carboxyl groups. Examples which may be mentioned are: tartaric acid, citric acid, malic acid; trimethylolpropane, trimethylolethane; pentaerythritol; polyether triols; glycerol; trimesic acid; trimellitic acid and its anhydride; pyromellitic acid and its dianhydride, and hydroxyisophthalic acid. If desired, compounds c7 are generally employed in amounts of from 0.01 to 15, preferably 0.05 to 10, especially preferably 0.1 to 4 mol %, based on component A.

Substances which can be employed as component c8 are aromatic or aliphatic diisocyanates. However, isocyanates with higher functionality may also be used. Examples of aromatic diisocyanates are toluylene 2,4-diisocyanate, toluylene 2,6-diisocyanate, 2,2′-diphenylmethane diisocyanate, 2,4′-diphenylmethane diisocyanate, 4,4′-diphenylmethane diisocyanate, naphthylene 1,5-diisocyanate or xylylene diisocyanate. Examples of aliphatic diisocyanates are, mainly, linear or branched alkylene diisocyanates or cycloalkylene diisocyanates with 2 to 20 carbon atoms, preferably 3 to 12 carbon atoms, for example 1,6-hexamethylene diisocyanate, isophorone diisocyanate or methylenebis(4-isocyanatocyclohexane). Substances which are furthermore suitable as component c8 are tri(4-isocyanophenyl)methane and the cyanurates, ureth diones and biureths of the abovementioned diisocyanates.

If desired, component c8 is generally employed in amounts of from 0.01 to 5, preferably 0.05 to 4 mol %, especially preferably 0.1 to 4 mol %, based on the total of the molar amounts of A and B.

Divinyl ethers c9 which may be employed are, generally all customary and commercially available divinyl ethers. The following are preferably used: 1,4-butanediol divinyl ether, 1,6-hexanediol divinyl ether or 1,4-cyclohexanedimethanol divinyl ether or mixtures of these. Preferably, the divinyl ethers are employed in amounts of from 0.01 to 5, in particular from 0.2 to 4, % by weight, based on the total weight of A and B.

The copolyesters are known, for example from WO96/15173 and WO 04/67632, or can be prepared by methods known per se.

The preferred copolyesters have a number-average molecular weight (Mₙ) in the range of from 1000 to 100 000, in particular in the range of from 9000 to 75 000 g/mol, preferably in the range of from 30 000 to 80 000 g/mol. They preferably have a melting point in the range of from 60 to 170, in particular in the range of from 60 to 150, ° C.

The abovementioned copolyesters may have hydroxyl and/or carboxyl end groups in any desired ratio. The abovementioned copolyesters may also be end-group-modified. Thus, for example, OH end groups may be acid-modified by reaction with phthalic acid, phthalic anhydride, trimellitic acid, trimellitic anhydride, pyromellitic acid or pyromellitic anhydride.

In one embodiment of the present invention, polymers P which may be employed may also be mixtures of various ester group-comprising polymers P, for example mixtures of the abovementioned copolyesters with polycaprolactones or polylactides, and mixtures of the ester-group-comprising polymers P with other biopolymers such as starch or with modified biodegradable biopolymers, such as modified starch, cellulose esters (for example cellulose acetate, cellulose acetate butyrate) or biodegradable artificial polymers such as polylactide (for example obtainable as EcoPLA® (Cargill)).

In a preferred embodiment of the present invention, the polymer P is selected from among polylactides, polycaprolactone, block copolymers of polylactide with poly-C₂-C₄-alkylene glycols, specifically with polyethylene glycol, and block copolymers of polycaprolactone with poly-C₂-C₄-alkylene glycols, specifically with polyethylene glycol.

Especially preferred polymers P are polycaprolactones, in particular those with a number-average molecular weight in the range of from 50 000 to 100 000 g/mol, especially preferably those with a number-average molecular weight in the range of from 80 000-100 000 g/mol. Especially preferred polymers P are also block copolymers of polycaprolactone with poly-C₂-C₄-alkylene glycols, specifically with polyethylene glycol, in particular those with a number-average molecular weight in the range of from 50 000 to 100 000 g/mol, especially preferably those with a number-average molecular weight in the range of from 80 000 to 100 000 g/mol.

The amount of polymer P will, as a rule, be selected in such a way that disintegration of the polymer particles is only achieved by the enzymatic degradation of the polymer, i.e. above the desired temperature upon exposure to moisture. Accordingly, the amount of polymer P is, as a rule, at least 20% by weight, frequently at least 30% by weight, preferably at least 40% by weight, in particular at least 45% by weight, and particularly preferably at least 50% by weight, based on the total mass of the components which form the active substance particles. The upper limit of the polymer P-content will naturally be limited by the other components which must be present and will, accordingly, not exceed 99% by weight, in particular 95% by weight and specifically 94% by weight, based on the total amount of the components which form the active substance particles. As a rule, the polymer P-content is in the range of from 20 to 99% by weight, frequently in the range of from 30 to 95% by weight, preferably in the range of from 40 to 95% by weight, in particular in the range of from 45 to 94% by weight and specifically in the range of from 50 to 90% by weight, or in the range of from 50 to 89% by weight or in the range of from 50 to 80% by weight, in each case based on the total amount of the components of the active substance-containing particles.

In the present application, the term “total amount”, which refers to the constituents in the active substance composition, corresponds to the total weight of the components of the active substance composition. The term “components of the active substance particles” corresponds to the term “components of the active substance composition”.

In accordance with the invention, the active substance compositions comprise at least one plant protectant. In the present context, the term “plant protectant” is to be understood in the broad sense and comprises not only substances which protect plants from attack by harmful organisms, substances which destroy plant-injurious organisms or which prevent their development, and substances which regulate the growth of the useful plant, i.e. which promote or reduce growth, including substances which serve to improve plant health. The plant protectants include, for example,
insecticides, acaricides and nematicides, i.e. active substances which destroy plant-injurious arthropods or nematodes or which reduce their development in such a manner that an attack of the useful plant is efficiently prevented or that the attack of a plant by these harmful organisms is reduced;
fungicides, i.e. active substances which destroy phytopathogenic fungi or which prevent their growth or which reduce the attack of the useful plant by such phytopathogenic fungi;
herbicides, i.e. active substances which destroy a harmful plant or which reduce or prevent their growth;
growth regulators, i.e. active substances which promote or reduce plant growth;
safeners, i.e. active substances which reduce or prevent a phytotoxic effect on the useful plant, which effect is triggered by the abovementioned substances; and
fertilizers.

Preferably, the plant protectant is an organic plant protectant, in particular a low-molecular-weight organic plant protectant with a molecular weight in the range of from 150 to 500 daltons.

Preferably, the plant protectant is solid or a nonvolatile oil at room temperature, i.e. it has a vapor pressure of less than 0.1 mbar at 25° C.

Examples of suitable plant protectants are listed hereinbelow. Examples of active substances with insecticidal, acaricidal and/or nematicidal activity are mentioned in groups A.1 to 15:
A.1. organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxime, pirimiphos-methyl, profenofos, prothiofos, sulprophos, tetrachlorvinphos, terbufos, triazophos, trichlorfon;
A.2. carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
A.3. pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, gamma-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
A.4. growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: jyriproxyfen, methoprene, fenoxycarb; d) lipid biosynthesis inhibitors: spirodiclofen, spiromesifen, spirotetramate;
A.5. compounds of nicotine receptor agonists/antagonists: clothianidin, dinotefuran, imidacloprid, thiamethoxam, nitenpyram, acetamiprid, thiacloprid; the thiazole compound of the formula (Γ¹)

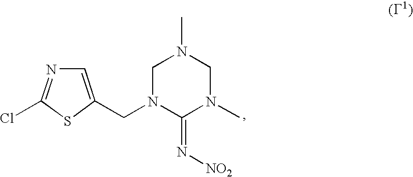

A.6. GABA-antagonist compounds: acetoprole, endosulfan, ethiprole, fipronil, vaniliprole, pyrafluprole, pyriprole,
   the phenylpyrazole compound of the formula Γ²

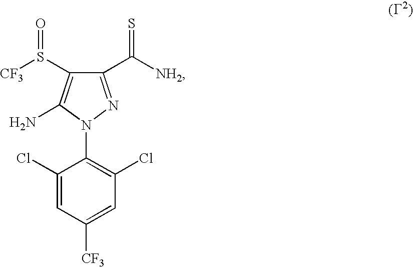

A.7. insecticidal macrocyclic lactones: abamectin, emamectin, milbemectin, lepimectin, spinosad,
   the compound of the formula (Γ³) (CAS No. 187166-40-1)

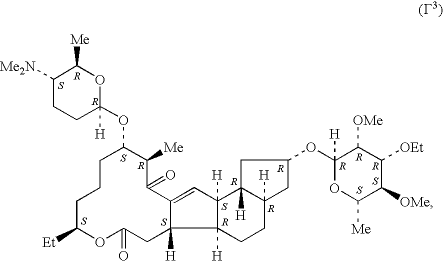

A.8. METI I compounds: fenazaquin, pyridaben, tebufenpyrad, tolfenpyrad, flufenerim;
A.9. METI II and III compounds: acequinocyl, fluacyprim, hydramethylnon;
A.10. decoupler compounds: chlorfenapyr;
A.11. compounds which act as oxidative phosphorylation inhibitors: cyhexatin, diafenthiuron, fenbutatin oxide, propargite;
A.12. molting inhibitor compounds: cyromazine;
A.13. mixed function oxidase inhibitor compounds: piperonyl butoxide;
A.14. sodium channel blocker compounds: indoxacarb, metaflumizone;
A.15. various: benclothiaz, bifenazate, cartap, flonicamid, pyridalyl, pymetrozine, sulfur, thiocyclam, flubendiamid, cyenopyrafen, flupyrazofos, cyflumetofen, amidoflumet; the aminoquinazoline compound of the formula (Γ⁴)

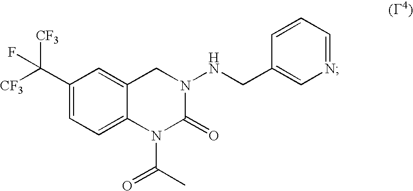

N-R′-2,2-dihalo-1-R″cyclopropanecarboxamide-2-(2,6-dichloro-α,α,α-trifluoro-p-tolyl)hydrazone or N-R′-2,2-di(R′″)propionamide-2-(2,6-dichloro-α,α,α-trifluoro-p-tolyl)hydrazone, where R′ is methyl or ethyl, halo is chlorine or bromine, R″ is hydrogen or methyl and R′″ is methyl or ethyl;
anthranilamide compounds of the formula (Γ⁵)

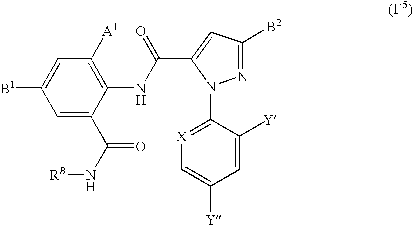

where A¹ is CH₃, Cl, Br or I, X is C—H, C—Cl, C—F or N, Y′ is F, Cl or Br, Y″ is H, F, Cl or CF₃, B¹ is hydrogen, Cl, Br, I or CN, B² is Cl, Br, CF₃, OCH₂CF₃ or OCF₂H and R^{B} is hydrogen, CH₃ or CH(CH₃)₂ and malonitrile compounds as described in JP 2002 284608, WO 02/89579, WO 02/90320, WO 02/90321, WO 04/06677, WO 04/20399, JP 2004 99597, WO 05/68423, WO 05/68432 or WO 05/63694, specifically the malonitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H and CF₃CF₂CH₂C(CN)₂CH₂(CF₂)₃CF₂H.
The commercially available compounds of group A can be found, among other publications, in The Pesticide Manual, 13^{th} Edition, British Crop Protection Council (2003). Thioamides of the formula Γ² and their synthesis have been described in WO 98/28279. Lepimectin is known from Agro Project, PJB Publications Ltd, November 2004. Benclothiaz and its synthesis have been described in EP-A1 454621. Methidathion and paraoxon and their synthesis have been described in Farm Chemicals Handbook, Volume 88, Meister Publishing Company, 2001. Acetoprole and its synthesis have been described in WO 98/28277. Metaflumizone and its synthesis have been described in EP-A1 462 456. Flupyrazofos has been described in Pesticide Science 54, 1988, p. 237-243 and in U.S. Pat. No. 4,822,779. Pyrafluprole and its synthesis have been described in JP 2002193709 and in WO 01/00614. Pyriprole and its synthesis have been described in WO 98/45274 and in U.S. Pat. No. 6,335,357. Amidoflumet and its synthesis have been described in U.S. Pat. No. 6,221,890 and in JP 21010907. Flufenerim and its synthesis have been described in WO 03/007717 and in WO 03/007718. Cyflumetofen and its synthesis have been described in WO 04/080180. Anthranilamides of the formula Γ⁵ and their synthesis have been described in WO 01/70671; WO 02/48137; WO 03/24222, WO 03/15518, WO 04/67528; WO 04/33468 and WO 05/118552. The malonitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H and CF₃CF₂CH₂C(CN)₂CH₂(CF₂)₃CF₂H have been described in WO 05/63694.

Examples of active substances with fungicidal activity are mentioned in groups B.1 to B.6:
B.1. strobilurins such as, for example, azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]-benzyl)carbamate, methyl(2-chloro-5-[1-(6-methylpyridin-2-ylmethoxyimino)-ethyl]benzyl)carbamate, methyl 2-(ortho-((2,5-dimethylphenyloxymethylene)-phenyl)-3-methoxyacrylate;
B.2 carboxamides such as, for example,
   carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide, 2-amino-4-methylthiazole-5-carboxanilide, 2-chloro-N-(1,1,3-trimethylindan-4-yl)nicotinamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(3′,4′,5′-trifluorobiphenyl-2-yl)-3-difluoromethyl-1-methyl-1H-pyrazole-4-carboxamide, N-(2′,4′,5′-trifluorobiphenyl-2-yl)-3-difluoromethyl-1-methyl-1H-pyrazole-4-carboxamide, N-[2-(1,1,2,3,3,3-hexafluoropropoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(3′,4′-dichloro-5-fluorobiphenyl-2-yl)-3-difluoromethyl-1-methyl-1H-pyrazole-4-carboxamide, N-(2-(1,3-dimethylbutyl)phenyl)-1,3,3-trimethyl-5-fluoro-1H-pyrazole-4-carboxamide, N-(4′-chloro-3′,5′-difluorobiphenyl-2-yl)-3-difluoromethyl-1-methyl-1H-pyrazole-4-carboxamide, N-(4′-chloro-3′,5′-difluorobiphenyl-2-yl)-3-trifluoromethyl-1-methyl-1H-pyrazole-4-carboxamide, N-(3′,4′-dichloro-5′-fluorobiphenyl-2-yl)-3-trifluoromethyl-1-methyl-1H-pyrazole-4-carboxamide, N-(3′,5′-difluoro-4′-methylbiphenyl-2-yl)-3-difluoromethyl-1-methyl-1H-pyrazole-4-carboxamide, N-(3′,5′-difluoro-4′-methylbiphenyl-2-yl)-3-trifluoromethyl-1-methyl-1H-pyrazole-4-carboxamide, N-(2-bicyclopropyl-2-yl-phenyl)-3-difluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoromethyl-1-methyl-1H-pyrazole-4-carboxamide;
   carboxylic acid morpholides: dimethomorph, flumorph;
   benzamides: flumetover, fluopicolid (picobenzamid), zoxamid;
   other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;
B.3. azoles such as, for example,
   triazoles: bitertanol, bromuconazole, cyproconazole, difenoconazole, diniconazole, enilconazole, epoxiconazole, fenbuconazole, flusilazol, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole, azaconazole, diniconazole-M, oxpoconazole, paclobutrazole, uniconazole, 1-(4-chlorophenyl)-2-([1,2,4]triazole-1-yl)cycloheptanol;
   imidazoles: cyazofamid, imazalil, pefurazoate, prochloraz, triflumizol;
   benzimidazoles: benomyl, carbendazim, fuberidazole, thiabendazole;
   others: ethaboxam, etridiazole, hymexazol;
B.4. nitrogen-containing heterocyclyl compounds such as, for example,
   pyridines: fuazinam, pyrifenox, 3-[5-(4-chlorophenyl)-2,3-dimethylisoxazolidin-3-yl]pyridine;
   pyrimidines: bupirimate, cyprodinil, ferimzone, fenarimol, mepanipyrim, nuarimol, pyrimethanil;
   piperazines: triforine;
   pyrroles: fludioxonil, fenpiclonil;
   morpholines: aldimorph, dodemorph, fenpropimorph, tridemorph;
   dicarboximides: iprodione, procymidone, vinclozoline;
   others: acibenzolar-S-methyl, anilazine, captan, captafol, dazomet, diclomezin, fenoxanil, folpet, fenpropidine, famoxadon, fenamidon, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindol-1-sulfonyl)-[1,2,4]triazole-1-sulfonamide;
B.5. carbamates and dithiocarbamates such as, for example,
   dithiocarbamates: ferbam, mancozeb, maneb, metiram, metam, propineb, thiram, zineb, ziram;
   carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
6. other fungicides such as, for example,
   guanidines: dodine, iminoctadine, guazatine;
   antibiotics: kasugamycin, polyoxines, streptomycin, validamycin A;
   organometal compounds: fentin salts;
   sulfur-containing heterocyclyl compounds: isoprothiolane, dithianon;
   organophosphorus compounds: edifenphos, fosetyl, fosetyl-aluminum, iprobenfos, pyrazophos, tolclofos-methyl, phosphorous acid and its salts;
   organochlorine compounds: thiophanate-methyl, chlorothalonil, dichlofluanid, tolylfluanid, flusulfamid, phthalide, hexachlorobenzene, pencycuron, quintozene; nitrophenyl derivatives: binapacryl, dinocap, dinobuton;
   inorganic active substances: Bordeaux mixture, copper acetate, copper hydroxide, copper oxychloride, basic copper sulfate, sulfur;
   others: spiroxamine, cyflufenamid, cymoxanil, metrafenon.

Examples of active substances with herbicidal activity are mentioned in groups C.1 to C.15:
C.1 lipid biosynthesis inhibitors such as, for example, chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop, alloxydim, butroxydim, clethodim, cloproxydim, cycloxydim, profoxydim, sethoxydim, tepraloxydim, tralkoxydim, butylate, cycloate, di-allate, dimepiperate, EPTC, esprocarb, ethiolate, isopolinate, methiobencarb, molinate, orbencarb, pebulate, prosulfocarb, sulfallate, thiobencarb, tiocarbazil, triallates, vernolate, benfuresate, ethofumesate and bensulid;
C.2 ALS-inhibitors such as, for example, amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac;
C.3 photosynthesis inhibitors such as, for example, atratone, atrazin, ametryn, aziprotryn, cyanazine, cyanatryn, chlorazine, cyprazine, desmetryn, dimethametryn, dipropetryn, eglinazine, ipazine, mesoprazine, methometon, methoprotryn, procyazine, proglinazine, prometon, prometryn, propazine, sebuthylazine, secbumeton, simazine, simeton, simetryn, terbumeton, terbuthylazine, tterbutryn, trietazine, ametridion, amibuzin, hexazinon, isomethiozin, metamitron, metribuzin, bromacil, isocil, lenacil, terbacil, brompyrazon, chloridazon, dimidazon, desmedipham, phenisopham, phenmedipham, phenmedipham-ethyl, benzthiazuron, buthiuron, ethidimuron, isouron, methabenzthiazuron, monoisouron, tebuthiuron, thiazafluoron, anisuron, buturon, chlorbromuron, chloreturon, chlorotoluron, chloroxuron, difenoxuron, dimefuron, diuron, fenuron, fluometuron, fluothiuron, isoproturon, linuron, methiuron, metobenzuron, metobromuron, metoxuron, monolinuron, monuron, neburon, parafluoron, phenobenzuron, siduron, tetrafluoron, thidiazuron, cyperquat, diethamquat, difenzoquat, diquat, morfamquat, paraquat, bromobonil, bromoxynil, chloroxynil, iodobonil, ioxynil, amicarbazon, bromofenoxim, flumezin, methazole, bentazone, propanil, pentanochlor, pyridates and pyridafol;
C.4 protoporphyrinogen-IX oxidase inhibitors such as, for example, acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidonethyl, flumiclorac, flumioxazin, flumipropyn, fluthiacet, thidiazimin, oxadiazone, oxadiargyl, azafenidin, carfentrazone, sulfentrazone, pentoxazone, benzfendizone, butafenacil, pyraclonil, profluazol, flufenpyr, flupropacil, nipyraclofen and etnipromid;
C.5 bleacher herbicides such as, for example, metflurazon, norflurazon, flufenican, diflufenican, picolinafen, beflubutamid, fluridon, fluorochloridon, flurtamon, mesotrione, sulcotrione, isoxachlortole, isoxaflutole, benzofenap, pyrazolynate, pyrazoxyfen, benzobicyclon, amitrole, clomazon, aclonifen, 4-(3-trifluoromethyl-phenoxy)-2-(4-trifluoromethylphenyl)pyrimidine and 3-heterocyclyl-substituted benzoyl derivatives of the formula II (see WO 96/26202, WO 97/41116, WO 97/41117 and WO 97/41118)

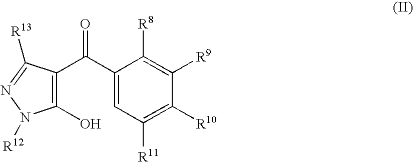

in which the variables R⁸ to R¹³ have the following meanings:
R⁸, R¹⁰ hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl or C₁-C₆-alkylsulfonyl;
R⁹ a heterocyclic radical selected from the group consisting of thiazol-2-yl, thiazol-4-yl, thiazol-5-yl, isoxazol-3-yl, isoxazol-4-yl, isoxazol-5-yl, 4,5-dihydroisoxazol-3-yl, 4,5-dihydroisoxazol-4-yl and 4,5-dihydroisoxazol-5-yl, where the nine radicals mentioned can be unsubstituted or mono- or polysubstituted, for example mono-, di-, tri- or tetrasubstituted by halogen, C₁-C₄-alkyl, C₁-C₄-alkoxy, C₁-C₄-haloalkyl, C₁-C₄-haloalkoxy or C₁-C₄-alkylthio;
R¹¹ hydrogen, halogen or C₁-C₆-alkyl;
R¹² C₁-C₆-alkyl;
R¹³ hydrogen or C₁-C₆-alkyl.
C.6 EPSP synthase inhibitors such as, for example, glyphosate;
C.7 glutamine synthethase inhibitors such as, for example, glufosinate and bilanaphos;
C.8 DHP synthase inhibitors such as, for example, asulam;
C.9 mitosis inhibitors such as, for example, benfluralin, butralin, dinitramin, ethalfluralin, fluchloralin, isopropalin, methalpropalin, nitralin, oryzalin, pendimethalin, prodiamin, profluralin, trifluralin, amiprofosmethyl, butamifos, dithiopyr, thiazopyr, propyzamid, tebutam, chlorthal, carbetamid, chlorbufam, chlorpropham and propham;
C.10 VLCFA inhibitors such as, for example, acetochlor, alachlor, butachlor, butena-chlor, delachlor, diethatyl, dimethachlor, dimethenamid, dimethenamid-P, metazachlor, metolachlor, S-metolachlor, pretilachlor, propachlor, propisochlor, prynachlor, terbuchlor, thenylchlor, xylachlor, allidochlor, CDEA, epronaz, diphenamid, napropamide, naproanilide, pethoxamid, flufenacet, mefenacet, fentrazamid, anilofos, piperophos, cafenstrole, indanofan and tridiphan;
C.11 cellulose biosynthesis inhibitors such as, for example, dichlobenil, chlorthiamid, isoxaben and flupoxam;
C.12 decoupler herbicides such as, for example, dinofenate, dinoprop, dinosam, dinoseb, dinoterb, DNOC, etinofen and medinoterb;
C.13 auxin herbicides such as, for example, clomeprop, 2,4-D, 2,4,5-T, MCPA, MCPA thioethyl, dichlorprop, dichlorprop-P, mecoprop, mecoprop-P, 2,4-DB, MCPB, chloramben, dicamba, 2,3,6-TBA, tricamba, quinclorac, quinmerac, clopyralid, fluoroxypyr, picloram, triclopyr and benazolin;
C.14 auxin transport inhibitors such as, for example, naptalam and diflufenzopyr;
C.15 benzoylprop, flamprop, flamprop-M, bromobutide, chlorflurenol, cinmethylin, methyldymron, etobenzanid, fosamine, metam, pyributicarb, oxaziclomefone, dazomet, triaziflam and methyl bromide.

Suitable safeners may be selected from the following enumeration:

benoxacor, cloquintocet, cyometrinil, dichlormid, dicyclonon, dietholate, fenchlorazole, fenclorim, flurazole, fluxofenim, furilazole, isoxadifen, mefenpyr, mephenate, naphthalic anhydride, 2,2,5-trimethyl-3-(dichloroacetyl)-1,3-oxazolidine (R-29148), 4-(dichloroacetyl)-1-oxa-4-azaspiro[4.5]decane (AD-67; MON 4660) and oxabetrinil. Examples of growth regulators are 1-naphthylacetamide, 1-naphthylacetic acid, 2-naphthyloxyacetic acid, 3-CPA, 4-CPA, ancymidol, anthraquinone, BAP, butifos; tribufos, butralin, chlorflurenol, chlormequat, clofencet, cyclanilide, daminozide, dicamba, dikegulac-sodium, dimethipin, chlorfenethol, etacelasil, ethephon, ethychlozate, fenoprop, 2,4,5-TP, fluoridamid, flurprimidol, flutriafol, gibberellic acid, gibberellin, guazatine, indolylbutyric acid, indolylacetic acid, karetazan, kinetin, lactidichlor-ethyl, maleic hydrazide, mefluidide, mepiquat-chloride, naptalam, paclobutrazole, prohexadione-calcium, quinmerac, sintofen, tetcyclacis, thidiazuron, triiodobenzoic acid, triapenthenol, triazethan, tribufos, trinexapac-ethyl, uniconazole.

Examples of fertilizers comprise potassium nitrate, potassium sulfate, urea, ammonium nitrate, monopotassium phosphate, ammonium phosphate, superphosphate, monoammonium phosphate, diammonium phosphate, ammonium polyphosphate, potassium dioxide, potassium chloride; calcium nitrate, calcium sulfate, calcium phosphate, magnesium sulfate, magnesium nitrate, magnesium lignosulfonates, ammonium sulfate, elemental sulfur, borax, sodium borate, copper sulfate, EDTA-Cu(NH₄)₂, EDTA-CuNa₂, iron oxide, iron dioxide, iron phosphate, iron sulfate, iron lignosulfonate, EDTA-FeK, EDTA-FeNa₃H₂O, EDTA-FeNH₄NH₄OH, DTPA-FeNa₂, DTPA-Fe(NH₄)₂, DTPA-FeNa₂, DTPA-FeHNa, HEDTA-Fe, HEDTA-Fe, HEDTA-Fe, EDDHA-FeNa, EDDHA-FeNa, manganese sulfate, manganese chloride, manganese oxide, manganese lignosulfonate, or manganese chelates, such as EDTA-MnK₂, EDTA-MnNa₂, sodium molybdate, potassium molybdate, zinc oxide, zinc sulfate, zinc oxysulfate, zinc lignosulfonate, zinc chelates such as EDTA-Zn(NH₄)₂, EDTA-ZnNa₂, selenium dioxide, selenium phosphate or selenium chloride.

With a view to the treatment of seeds, the plant protectants are preferably selected among organic active substances which have a fungicidal, insecticidal, acaricidal and/or nematicidal activity. In particular, they are one or more of the following substances:

Substances with insecticidal or acaricidal or nematicidal activity, which are selected in particular from acetamiprid, alpha-cypermethrin, beta-cypermethrin, bifenthrin, carbofuran, carbosulfan, clothianidin, cycloprothrin, cyfluthrin, cypermethrin, deltamethrin, diflubenzuron, dinotefuran, etofenprox, fenbutatin oxide, fenpropathrin, fipronil, flucythrinate, imidacloprid, lambda-cyhalothrin, nitenpyram, pheromones, spinosad, teflubenzuron, tefluthrin, terbufos, thiacloprid, thiamethoxam, thiodicarb, tralomethrin, triazamate, zeta-cypermethrin, spirotetramate, flupyrazofos, NC 512, tolfenpyrad, flubendiamide, bistrifluoron, benclothiaz, DPX-E2Y45, HGW86, pyrafluprol, pyriprol, F-7663, F-2704, amidoflumet, flufenerim and cyflumetofen.

Substances with fungicidal activity, for example metalaxyl, oxadixyl, guazatine, pyrimethanil, streptomycin, fungicides from group B.3, in particular triazoles such as, for example, difenoconazole, epoxiconazole, fluquiconazole, flutriafol, hymexazol, imazalil, metconazole, prochloraz, prothioconazole, tebuconazole, thiabendazole, triadimenol or triticonazole, furthermore iprodion, maneb, mancozeb, metiram, thiram, benomyl, boscalid, carbendazim, carboxin, dazomet, silthiofam, copper fungicides, fludioxonil, sulfur, dazomet, fungicides of group B1, in particular azoxystrobin, kresoxim-methyl, orysastrobin, pyraclostrobin or trifloxystrobin, and captan or dimethomorph.

The abovementioned plant protectants may be employed alone or in combination with one another.

The total amount of plant protectant in the active substance-containing particles is typically in a range of from 1 to 30% by weight, frequently in the range of from 5 to 30% by weight, in particular in the range of from 8 to 25% by weight, based on the total amount of the components of the active substance particles. The ratio of polymer P to plant protectant is preferably at least 1:1 and is in particular in the range of from 2:1 to 10:1.

A preferred embodiment of the present invention are active substance compositions in which the at least one plant protectant comprises at least one fungicide, in particular at least one of the preferred fungicides and in particular at least one of the azole fungicides referred to as group B.3, and in particular is selected from among these. In this preferred embodiment, the polymer P is preferably selected among polylactides, polycaprolactone, block copolymers of polylactide with poly-C₂-C₄-alkylene glycols, specifically with polyethylene glycol, and block copolymers of polycaprolactone with poly-C₂-C₄-alkylene glycols, specifically with polyethylene glycol. Especially preferred polymers P of this embodiment are polycaprolactones, in particular those with a number-average molecular weight in the range of from 50 000 to 100 000 g/mol, especially preferably those with a number-average molecular weight in the range of from 80 000 to 100 000 g/mol. Especially preferred polymers P of this embodiment are also block copolymers of polycaprolactone with poly-C₂-C₄-alkylene glycols, specifically with polyethylene glycol, in particular those with a number-average molecular weight in the range of from 50 000 to 100 000 g/mol, especially preferably those with a number-average molecular weight in the range of from 80 000 to 100 000 g/mol.

The type of the enzyme present in the active substance composition depends, in a manner known per se, on the type of the enzymatically degradable polymer P. In accordance with the invention, the at least one enzyme is a hydrolase, i.e. an enzyme which is capable of hydrolytically, that is by addition of water, cleaving chemical bonds.

Examples of suitable enzyme classes are detailed hereinbelow:

enzymes which are capable of hydrolyzing ester bonds (esterases: enzyme class
EC 3.1) such as enzymes of the EC classes
EC 3.1.1 (carboxylic ester hydrolases),
EC 3.1.2 (thioester hydrolases),
EC 3.1.3 (phosphoric acid monoester hydrolases),
EC 3.1.4 (phosphoric acid diester hydrolases),
EC 3.1.5 (triphosphoric acid monoester hydrolases),
EC 3.1.6 (sulfuric ester hydrolases),
EC 3.1.7 (diphosphoric monoester hydrolases),
EC 3.1.8 (phosphoric triester hydrolases);
glycosylases (EC 3.2), for example glycosylases from the EC classes
EC 3.2.1 (glycosidases, i.e. enzymes which hydrolyze O- and S-glycosyl compounds),
EC 3.2.2 (enzymes which hydrolyze N-glycosyl compounds),
EC 3.2.3 (enzymes which hydrolyze S-glycosyl compounds);
enzymes which hydrolyze ether bonds (EC 3.3), for example enzymes from the EC classes
EC 3.3.1 (thioether and trialkylsulfonium hydrolases)
EC 3.3.2 (ether hydrolases)
enzymes which hydrolyze peptide bonds, i.e. peptidases (EC 3.4), for example peptidases from the EC classes
EC 3.4.11 (aminopeptidases)
EC 3.4.13 (dipeptidases)
EC 3.4.14 (dipeptidylpeptidases and tripeptidylpeptidases)
EC 3.4.15 (peptidyldipeptidases)
EC 3.4.16 (serine type carboxypeptidases)
EC 3.4.17 (metallocarboxypeptidases)
EC 3.4.18 (cysteine-type carboxypeptidases)
EC 3.4.19 (omega peptidases)
EC 3.4.21 (serine endopeptidases)
EC 3.4.22 (cysteine endopeptidases)
EC 3.4.23 (aspartic endopeptidases)
EC 3.4.24 (metalloendopeptidases)
EC 3.4.25 (threonine endopeptidases)
EC 3.4.99 (endopeptidases with unknown catalytic mechanism)
enzymes which hydrolyze carbon-nitrogen bonds which are not amide bonds (EC 3.5), for example enzymes from the EC classes
EC 3.5.1 (enzymes which catalyze the hydrolysis of linear amides)
EC 3.5.2 (enzymes which catalyze the hydrolysis of cyclic amides)
EC 3.5.3 (enzymes which catalyze the hydrolysis of linear amidines)
EC 3.5.4 (enzymes which catalyze the hydrolysis of cyclic amidines)
EC 3.5.99 (enzymes which catalyze the hydrolysis of other compounds)
enzymes which hydrolyze acid anhydrides (EC3.6), for example enzymes of the EC classes
EC 3.6.1 (enzymes which catalyze the hydrolysis of phosphorus-comprising anhydrides)
EC 3.6.2 (enzymes which catalyze the hydrolysis of sulfonyl-comprising anhydrides)
EC 3.6.3 (enzymes which are catalytically active on acid anhydrides)
EC 3.6.4 (enzymes which are catalytically active on acid anhydrides)
EC 3.6.5 (enzymes which are catalytically active on GPT)
enzymes which hydrolyze carbon-carbon bonds (EC 3.7), for example
EC 3.7.1 (enzymes which catalyze the hydrolysis of ketone-containing substrates)
enzymes which hydrolyze halogen-carbon bonds (EC 3.8), for example enzymes from the EC class
EC 3.8.1 (enzymes which hydrolyze C-halogen compounds)
enzymes which hydrolyze phosphorus-nitrogen bonds (EC3.9)
enzymes which hydrolyze sulfur-nitrogen bonds (EC3.10)
enzymes which hydrolyze carbon-phosphorus bonds (EC3.11)
enzymes which hydrolyze sulfur-sulfur bonds (EC3.12) and
enzymes which hydrolyze carbon-sulfur bonds (EC3.13).

Among the abovementioned enzymes, those from the group of the amidases (EC 3.5), the proteases (EC 3.4) and the esterases (EC 3.1) are preferred.

If the polymer P is a polymer with a multiplicity of ester groups in the polymer backbone, the enzyme will, as a rule, be an esterase (enzyme class EC 3.1.X.X), in particular a carboxylic ester hydrolase (enzyme class EC 3.1.1.X), specifically
an enzyme from the group of the lipases (EC 3.1.1.3, triacylglycerol lipase), for example a lipase from Aspergillus oryzae as is available for example from Novozyme under the name Novozymes CaLB L, a lipase from Burkholderia plantarii (=Burkholderia glumae), as is described for example in J. prakt. Chem., 1997, 339, p. 381-384 or under Swiss-Prot. No. Q05489 (UniProtKB/Swiss-Prot entry), a lipase B from Candida antarctica as is described, for example, in Structure 1994, 2, p. 293-298 or in Biochemistry 24, 1995, p. 16838-16851, or
an enzyme from the group of the cutinases (EC 3.1.1.74), for example a cutinase from Fusarium solani, for example cutinase 1 from Fusarium solani subsp. pisi (Nectria haematococca), as is described, for example, in Nature, 1992, 356, p. 615-618.

In an especially preferred embodiment of the invention, the enzyme is a lipase from Candida antarctica, for example the lipase described in Structure 1994, 2, p. 293-298 or in Biochemistry 24, 1995, p. 16838-16851.

In another preferred embodiment of the invention, the enzyme is a lipase from Burkholderia plantarii, for example a lipase (=Burkholderia glumae), as is described, for example, in J. prakt. Chem., 1997, 339, p. 381-384 or under Swiss-Prot. No. Q05489 (UniProtKB/Swiss-Prot entry).

Others which are suitable are comparable enzymes of synthetic or natural origin, for example modified lipases. Examples of modified enzymes are those with an increased activity at low temperatures (cryophilic enzymes), for example at temperatures in the range of from 10 to 25° C. The enzymes used may also be recombinant enzymes, that is to say enzymes which have been prepared with the aid of genetically modified organisms. Such enzymes also comprise homologs of the authentic enzyme, for example versions with an increased stability to chemical or thermal denaturation, increased activity at low temperatures and the like. The enzymes which are suitable in accordance with the invention also comprise those enzymes which have been subjected to a post-translational modification.

In an especially preferred embodiment of the invention, the enzyme is a lipase (EC 3.1.1.3, triacylglycerol lipase). In this preferred embodiment, the polymer P is preferably selected among polylactides, polycaprolactone, block copolymers of polylactide with poly-C₂-C₄-alkylene glycols, specifically with polyethylene glycol, and block copolymers of polycaprolactone with poly-C₂-C₄-alkylene glycols, specifically with polyethylene glycol. Especially preferred polymers P of this embodiment are polycaprolactones, in particular those with a number-average molecular weight in the range of from 50 000 to 100 000 g/mol, especially preferably those with a number-average molecular weight in the range of from 80 000 to 100 000 g/mol. Especially preferred polymers P of this embodiment are also block copolymers of polycaprolactone with poly-C₂-C₄-alkylene glycols, specifically with polyethylene glycol, in particular those with a number-average molecular weight in the range of from 50 000 to 100 000 g/mol, especially preferably those with a number-average molecular weight in the range of from 80 000 to 100 000 g/mol.

The enzyme selected will preferably be one which is essentially inactive at a temperature of below 10° C. Preferably, the enzyme selected will be one which has a hydrolase activity at a temperature of from 15 to 20° C. which is sufficient for degrading the polymer. The selection of suitable enzymes can be accomplished by the skilled worker on the basis of his expert knowledge and assays for determining the temperature and substrate specificity of the hydrolase activity.

In the case of carboxylesterases, the temperature specificity of the esterase activity can be determined for example by using the assay described in the examples, where p-nitrophenyl acetate is enzymatically hydrolyzed at the test temperature using the test enzyme, and the amount of p-nitrophenol us subsequently determined by HPLC. A sufficient activity is, as a rule, provided when the enzyme has an activity of 100 U/mg (based on the hydrolysis of p-nitrophenylacetate) at the desired temperature. The activity of the hydrolase toward the polymer P can be estimated for example via an assay in which the decrease of the pH of a buffered suspension of the polymer P, which comprises the enzyme, is determined. An example for such an assay is detailed in the examples.

Naturally, the amount of hydrolase in the active substance particles according to the invention depends on the activity of the hydrolase toward the enzymatically degradable polymer. It is typically in the range of from 0.1 to 10% by weight, in particular in the range of from 0.5 to 8% by weight and specifically in the range of from 1 to 5% by weight, based on the total amount of the components of the active substance particles.

In addition to the abovementioned components, the active substance particles of the active substance compositions according to the invention may also comprise further components in an amount of up to 70% by weight, frequently up to 60% by weight, in particular up to 50% by weight or up to 40% by weight, specifically in an amount of up to 35%, based on the total amount of the active substance particles. These include in particular components which are conventionally employed in the preparation of powders, and water-soluble polymers. The amount of the water-soluble polymers will, as a rule, not exceed 70% by weight, frequently 60% by weight, in particular 50% by weight or 40% by weight, specifically 35% by weight and very specifically 30% by weight, based on the total amount of the active substance particles and is, if desired, as a rule at least 0.5% by weight or at least 1% by weight, frequently at least 2% by weight, in particular at least 5% by weight, preferably at least 10% by weight or at least 15% by weight, based on the total amount of the active substance particles. If desired, the amount of the water-soluble polymers is, as a rule, in the range of from 1 to 70% by weight, frequently in the range of from 2 to 60% by weight, in particular in the range of from 5 to 50% by weight or 10 to 40% by weight or in the range of from 0.5 to 40% by weight, preferably in the range of from 1 to 35% by weight and specifically in the range of from 5 to 30% by weight, based on the total amount of the active substance particles. It may also be advantageous to employ larger amounts of water-soluble polymers, for example from 5 to 70% by weight, in particular from 10 to 65% by weight or 20 to 60% by weight, based on the total amount of the active substance particles.

The water-soluble polymers bring about good resuspendability of the active substance particles according to the invention in water, which may be helpful in particular in the treatment of seed. A premature release of the active substance does not take place, or takes place only to a limited extent.

Examples of water-soluble polymers are polyvinylpyrrolidones, copolymers of vinylpyrrolidone, in particular those having a vinylpyrrolidone content of at least 50% by weight, in particular at least 70% by weight, e.g. vinylpyrrolidone/C₁-C₄-alkyl (meth)acrylate copolymers and vinylpyrrolidone/vinyl acetate copolymers, polyvinylformamides, partially hydrolyzed polyvinylformamides, in particular those with a degree of hydrolysis in the range of from 10 to 99%, homo- and copolymers of acrylic acid, in particular those with an acrylic acid content of at least 20% by weight, homo- and copolymers of methacrylic acid, in particular those with a methacrylic acid content of at least 20% by weight, homo- and copolymers of acrylamide, in particular those with an acrylamide content of at least 40% by weight, polyethylenimines, polyvinylamines, polycaprolactams, polyvinyl alcohols, partially hydrolyzed polyvinyl acetates with a degree of hydrolysis of >50%, cellulose, cellulose derivatives such as hydroxyalkylcelluloses, alkylhydroxyalkylcelluloses, carboxyalkylcelluloses, alkylhydroxyalkylcellulose acetate succinates, alkylhydroxyalkyl cellulose acetate phthalates, alkylhydroxyalkylcellulose phthalates, cellulose acetate phthalates, modified starches and starch derivatives such as hydroxylalkyl starches, carboxyalkyl starches, roasted starches, oxidized starches, octenylsuccinate starches and the like, dextrans and poly-C₂-C₃-oxyalkylenes such as polyethylene oxide, polypropylene oxide and polyethylene oxide/polypropylene oxide block copolymers.

In accordance with a preferred embodiment, the water-soluble polymer is selected among polyvinylpyrrolidones and copolymers of vinylpyrrolidone, in particular those with a vinylpyrrolidone content of at least 50% by weight, in particular at least 70% by weight. In this preferred embodiment, the polymer P is preferably selected among polylactides, polycaprolactone, block copolymers of polylactide with poly-C₂-C₄-alkylene glycols, specifically with polyethylene glycol, and block copolymers of polycaprolactone with poly-C₂-C₄-alkylene glycols, specifically with polyethylene glycol. Especially preferred polymers P of this embodiment are polycaprolactones, in particular those with a number-average molecular weight in the range of from 50 000 to 100 000 g/mol, especially preferably those with a number-average molecular weight in the range of from 80 000 to 100 000 g/mol. Especially preferred polymers P of this embodiment are also block copolymers of polycaprolactone with poly-C₂-C₄-alkylene glycols, specifically with polyethylene glycol, in particular those with a number-average molecular weight in the range of from 50 000 to 100 000 g/mol, especially preferably those with a number-average molecular weight in the range of from 80 000 to 100 000 g/mol.

A specific embodiment of the invention relates to an active substance composition in which the active substance particles
a) at least one plant protectant from the group of the fungicides, in particular at least one plant protectant from the group of the azole fungicides (group B.3), as a rule in an amount of from 1 to 30% by weight, frequently from 5 to 30% by weight, in particular from 8 to 25% by weight, based on the total amount of the components of the active substance particles or the active substance composition;
b) at least one polymer P which is selected among polylactides, polycaprolactone, block copolymers of polylactide with poly-C₂-C₄-alkylene glycols, specifically with polyethylene glycol, and block copolymers of polycaprolactone with poly-C₂-C₄-alkylene glycols, specifically with polyethylene glycol, in particular at least one polymer P which is selected among polycaprolactones, in particular those with a number-average molecular weight in the range of from 50 000 to 100 000 g/mol, especially preferably those with a number-average molecular weight in the range of from 80 000 to 100 000 g/mol, and block copolymers of polycaprolactone with poly-C₂-C₄-alkylene glycols, specifically with polyethylene glycol, in particular those with a number-average molecular weight in the range of from 50 000 to 100 000 g/mol, especially preferably those with a number-average molecular weight in the range of from 80 000 to 100 000 g/mol, as a rule in an amount of from 20 to 99% by weight, frequently in the range of from 30 to 95% by weight, preferably in the range of from 40 to 95%, in particular in the range of from 45 to 94% by weight, specifically in the range of from 50 to 90% by weight and very specifically in the range of from 50 to 89% by weight or in the range of from 50 to 80% by weight, in each case based on the total amount of the components of the active substance-comprising particles;
c) at least one carboxylic ester hydrolase (EC 3.1.1) and in particular at least one lipase (EC 3.1.1.3) or at least one cutinase (EC 3.1.1.74) and especially preferably at least one of the lipases and/or cutinases which have been specified as being preferred, as a rule in an amount of from 0.1 to 10% by weight, in particular from 0.5 to 8% by weight and specifically from 1 to 5% by weight, based on the total amount of the components of the active substance particles;
d) at least one water-soluble polymer which is preferably selected among polyvinylpyrrolidones and copolymers of vinylpyrrolidone, in particular those with a vinylpyrrolidone content of at least 50% by weight, in particular at least 70% by weight, as a rule in an amount of from 0.5 to 70% by weight, frequently from 1 to 60% by weight, in particular from 2 to 50% by weight or 5 to 40% by weight, specifically from 10 to 35% by weight or 15 to 30% by weight, based on the total amount of the components of the active substance particles;
   where the total amount of component a), b) and c) accounts, as a rule, for from 30 to 99.5% by weight, frequently from 40 to 99% by weight, in particular from 50 to 98% by weight or from 60 to 95% by weight, specifically from 65 to 90% by weight or from 70 to 85% by weight, based on the total amount of the components of the active substance particles.

In addition, the compositions according to the invention may furthermore comprise other components as are suitable for the preparation of powder compositions of plant protectants. Examples are stabilizers, salts, buffers, anticaking agents and the like. As a rule, their content in the active substance composition will not exceed 20% by weight and in particular 10% by weight, and is, if desired, in the ranges required for achieving the desired effect, for example in the range of from 0.001 to 20% by weight or in the range of from 0.01 to 10% by weight based on the total amount of the components of the active substance particles.

According to the invention, the active substance composition is a powder in which at least 90% by weight of the particles a diameter of no more than 500 μm, in particular no more than 400 μm, preferably no more than 300 μm and specifically no more than 200 μm. As a rule, at least 90% by weight of the particles have a diameter in the range of from 0.1 to 500 μm, in particular in the range of from 0.2 to 400 μm, preferably in the range of from 0.3 to 300 μm and specifically in the range of from 0.5 to 200 μm. The determination of the particle diameters and the distribution of the particle diameters as discriminated by percent by weight can be effected in a manner known per se, for example by light scattering in a 1% by weight aqueous dispersion of the powder according to the invention, obtainable by diluting the powder with water. The mean diameter of the active substance particles (which can be determined as the Z mean by light scattering of a 1% by weight aqueous dispersion of the powder according to the invention) can vary within a wide range. In general, it amounts to at least 0.2 μm, preferably at least 0.3 μm, especially preferably at least 0.5 μm. The mean diameter is preferably in the range of from 0.2 to 450 μm, preferably 0.3 to 300 μm, in particular 0.5 to 200 μm.

The particles present in the active substance composition according to the invention can have the morphology which is customary for powders, including a core-shell morphology or microcapsule morphology. In contrast to microcapsules, however, they frequently have a compact structure, with the polymers being distributed essentially uniformly throughout the particle cross-section, it being possible for the active substance and/or the enzyme to show a concentration gradient within the particles or to be distributed uniformly.

The preparation of the active substance compositions according to the invention can be accomplished by customary methods for the preparation of pulverulent substances whose powder particles have in the stated range and comprise a plurality of components. As a rule, the components of the active substance-containing particles are mixed with one another and then processed by customary methods to give a finely divided powder. Such a process is also subject matter of the present application.

Examples of processes which are suitable in accordance with the invention are coprecipitation and drying methods such as spray drying, fluidized-bed drying, fluidized-bed coating, micronization, preparation of Pickering dispersions with subsequent spray drying, and the like.

Coprecipitation is described for example in WO99/00013, the disclosure of which is herewith referred to.

In accordance with a preferred embodiment of the invention, the preparation of the active substance composition according to the invention is accomplished by a spray-drying method.

To this end, in a first step, the components of the active substance-containing particles will be mixed with one another, or dissolved, in a suitable solvent or diluent. The resulting suspension or solution will subsequently be subjected to a spray-drying method. Here, the solvent or diluent is removed with the aid of a stream of warm gas, where the components of the active substance particles which are present in the solution or suspension form a finely divided powder which can be obtained in a manner known per se. As an alternative, the components of the active substance particles can be dissolved or dispersed separately and the resulting solutions or dispersions can be subjected to concomitant spray-drying.

In the preparation of the active substance composition according to the invention by a spray-drying method, the components of the active substance-containing particles will, in a first step, be dissolved or suspended in a suitable solvent or diluent. Preferred solvents are those in which all components of the active substance-containing particles dissolve and which do not destroy the hydrolase employed.

Examples of suitable solvents are:
aliphatic and alicyclic ethers with preferably 4 to 10 C atoms such as tetrahydrofuran, dioxane, diethyl ether, diisopropyl ether, methyl tert-butyl ether;
halohydrocarbons such as dichloromethane, trichloromethane, dichloroethane;
cyclic or open-chain carbonates such as ethylene carbonate, propylene carbonate, diethyl carbonate;
and mixtures of the abovementioned solvents and mixtures of the abovementioned solvents with water.

Also suitable is water as the only solvent or diluent.

In a second step, the solvent is subsequently removed in a suitable spray apparatus with the aid of a stream of warm gas. To this end, the solution(s) or dispersion(s) is/are sprayed into a stream of warm air in a suitable apparatus. Spraying in the solution(s) or dispersion(s) can be effected in cocurrent or in countercurrent with the stream of warm air, preferably in cocurrent, i.e. in the same direction as the stream of warm air.

Suitable apparatuses for spraying in are single- or multi-substance nozzles and atomizer disks.

The temperature of the stream of warm gas, hereinbelow also referred to as drying gas, is typically in the range of from 50 to 200° C., in particular in the range of from 70 to 180° C. and specifically in the range of from 100 to 160° C. upon entering into the drying apparatus. When the drying gas leaves the drying apparatus, its temperature is typically in the range of from 40 to 120° C. and in particular in the range of from 60 to 100° C. Suitable drying gases are, besides air, in particular inert gases such as nitrogen, argon or helium, with nitrogen being preferred. In the case of readily volatile solvents, it is also possible to employ lower temperatures, for example room temperature.

Typically, spray-drying is effected in spray-drying towers which are suitable for this purpose. Here, the solution(s) or dispersion(s) to be dried and the drying gas are typically introduced into the tower at the top. At the bottom of the tower, the dry active substance particles are discharged together with the gas stream and separated from the gas stream in apparatuses which are arranged downstream, such as cyclones. Besides conventional spray-drying, it is also possible to perform an agglomerating spray-drying operation using an internal or external fluidized bed (for example what is known as the FSD technology from Niro), where the particles formed agglomerate to give larger bodies. The primary particle size of the particles formed is, however, preferably in the abovementioned ranges and will in particular not exceed 300 μm and specifically 200 μm.

If appropriate, the active substance particles, in particular when they have a certain tackiness, will be provided with traditional spray-drying adjuvants. These are finely divided solids which are introduced into the spray-drying apparatus together with the solution(s) or dispersion(s) and which ensure that no agglutination or clumping takes place. Suitable finely divided solids are in particular silicas including hydrophobicized silica, alkali metal and alkaline earth metal silicates, alkaline earth metal alumosilicates, highly crosslinked polyvinylpyrrolidone, celluloses, starches, highly crosslinked sodium carboxymethyl starch or crosslinked sodium carboxymethylcellulose. The particle size of these substances is typically below 100 μm (D₉₀ value).

The active substance compositions which are obtainable in accordance with the invention can be employed in plant protection per se. Since, as a rule, they are dispersible in water, they may also be incorporated into liquid use forms such as, for example, dilute spray mixtures.

Depending on the active substance(s) present in the active substance particles, the active substance compositions according to the invention can be employed for combating harmful plants, phytopathogenic fungi, plant-injurious insects, acarids and nematodes, but also for controlling the growth of the useful plants.

The active substance compositions according to the invention are particularly suitable for the treatment of seed and of the soil.

In the case of the treatment of the soil, the composition according to the invention, if appropriate in the form of a dilute aqueous suspension of the active substance particles, will be introduced into the soil. As a rule, the introduction into the soil is accomplished before or after sowing the useful plants, preferably before the emergence of the useful plants.

In particular, the active substance compositions according to the invention are also suitable for the treatment of seed. To this end, the conventional, i.e. untreated, seed or else already pretreated seed will be treated with an active substance composition according to the invention or an aqueous population of the active substance composition which, in addition to water and the active substance-containing particles, may additionally comprise conventional constituents of seed-treatment products, such as, for example, adhesives, colorants, surface-active substances such as dispersants, furthermore organic and inorganic thickeners, bactericides, antifreeze agents, antifoams and the like.

Examples of colorants are not only pigments, which are sparingly soluble in water, but also dyes, which are water-soluble. Examples which may be mentioned are the dyes known by the names Rhodamin B, C.I. Pigment Red 112 and C.I. Solvent Red 1, and pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohols, partially hydrolyzed polyvinyl acetates and tylose.

Suitable surface-active agents (adjuvants, wetters, adhesives, dispersants and emulsifiers) are the alkali metal, alkaline-earth metal, ammonium salts of aromatic sulfonic acids, for example lignosulfonic acids (for example Borrespers types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl sulfonates and alkylaryl sulfonates, alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene, or of the naphthalenesulfonic acids, with phenol and formaldehyde, polyoxyethylene octylphenol ether, ethoxylated isooctyl-, octyl- or nonylphenol, alkylphenyl polyglycol ether, tributylphenyl polyglycol ether, alkylaryl polyether alcohols, isotridecyl alcohol, fatty alcohol/ethylene oxide condensates, ethoxylated castor oil, polyoxyethylene alkyl ether or polyoxypropylene alkyl ether, lauryl alcohol polyglycol ether acetate, sorbitol esters, lignin-sulfite waste liquors, and proteins, denatured proteins, polysaccharides (for example methyl-cellulose), hydrophobicized starches, polyvinyl alcohol (Mowiol types, Clariant), polycarboxylates (BASF AG, Sokalan types), polyalkoxylates, polyvinylamine (BASF AG, Lupamin types), polyethylenimine (BASF AG, Lupasol types), polyvinylpyrrolidone and their copolymers.

Examples of thickeners (i.e. compounds which impart a modified flowing behavior to the formulation, i.e. high viscosity in the state of rest and low viscosity in the state of motion) are polysaccharides such as xanthan gum (Kelzan® from Kelco), Rhodopol® 23 (Rhone Poulenc) or Veegum® (from R.T. Vanderbilt), and inorganic and organic layer minerals such as Attaclay® (from Engelhardt).

Examples of antifoams are silicone emulsions (such as, for example, Silikon® SRE, from Wacker or Rhodorsil® from Rhodia), long-chain alcohols, fatty acids, salts of fatty acids, for example magnesium stearate, organofluorine compounds and their mixtures.

Bactericides may be added for stabilization purposes. Examples of bactericides are bactericides based on dichlorophen and benzyl alcohol hemiformal (Proxel® from ICI or Acticide® RS from Thor Chemie and Kathon® MK from Rohm & Haas), and isothiazolinone derivatives such as alkylisothiazolinones and benzisothiazolinones (Acticide Mbs from Thor Chemie)

Antifreeze agents: for example C₁-C₄-alkanols such as ethanol, isopropanol, n-butanol, isobutanol, and C₂-C₆-polyols such as glycerol, ethylene glycol, hexylene glycol and/or propylene glycol.

Seed can be treated by the customary techniques for treating seed, for example by seed coating, seed dusting, seed soaking and seed dressing.

In accordance with a first embodiment of the seed treatment, the seed, i.e. the parts of the plants which are capable of propagation and which are intended for sowing, are treated with an active substance composition according to the invention or an aqueous preparation of the active substance composition according to the invention. In this context, the term seed encompasses kernels, seeds, fruits, tubers, slips or similar products, in particular kernels and seeds.

The treatment of the plant parts can be accomplished for example by mixing the plant parts with an aqueous suspension of the active substance composition according to the invention or by dusting the seed with a seed composition according to the invention. These measures can be carried out in specific apparatuses for the treatment of seed, for example in drill seeders. However, treatment is also possible in a simple manner by mixing an aqueous suspension of the active substance composition according to the invention with the seed in a container, for example in a bucket or a tray, and subsequently allowing the seed to dry.

As an alternative, it is also possible to treat the seed with the active substance composition according to the invention during sowing.

In a further embodiment of the seed or soil treatment according to the invention, the active substance composition according to the invention, if appropriate in the form of an aqueous suspension, will be introduced into furrows which already contain the seed. As an alternative, it is also possible to first treat the furrow of the field with the active substance composition according to the invention or an aqueous preparation thereof and then to introduce the seed into the furrow of the field.

Depending on the nature of the active substance employed, the active substance compositions according to the invention are suitable for treating the seed of any useful plants, for example cereal plants, root plants, oil plants, vegetables, spices, ornamentals and the like, for example for the treatment of seed of the following plants: durum wheat and other wheat species, oats, rye, barley, maize, including fodder maize and sweetcorn) soybeans, brassicas, cotton, sunflower, bananas, rice, oilseed rape, beet, sugar beet, fodder beet, eggplants, potatoes, turf, grass seed, tomatoes, leek, pumpkin, cabbage, salad plants, peppers, cucumbers, melons, beans, peas, garlic, onions, carrots, tobacco, grapes, petunias, geraniums, pelargoniums, pansies and the like. The active substance compositions according to the invention are also suitable for the treatment of the seed of transgenic crop plants which are resistant to herbicides, for example to sulfonylureas, imidazolinones, glufosinates, glyphosates, cyclohexadione/aryloxyphenoxypropionic acid herbicides, and for the treatment of seed which is suitable for producing Bt toxins (Bacillus thuringiensis toxins).

Preferably, the active substance compositions according to the invention will be employed in such an amount that the amount of active substance in the seed is in the range of from 0.1 g to 10 kg per 100 kg of seed, preferably in the range of from 1 g to 5 kg per 100 kg of seed, in particular in the range of from 1 g to 2.5 kg per 100 kg of seed. For certain plants such as salad plants and onions, it is also possible to choose a greater amount of active substance.

The seed which has been treated in accordance with the invention is distinguished by advantageous characteristics in comparison with conventionally treated seed and is therefore likewise subject matter of the present application.

As an alternative, spray applications on plants which have already grown are also feasible. To this end, the active substance compositions according to the invention can be applied to the plants as such, as a dilution with water or in the form of dilute formulations.

The compositions according to the invention may also be incorporated into active substance formulations which are then applied as such or in dilute form, for example as an aqueous spray mixture. Such formulations may be solid, semi-solid, for example powders, dusts, pastes, granules, or liquid, for example suspensions or dispersions, for example, aqueous, oil-based or other suspensions or dispersions. The formulations, or the spray mixtures which have been obtained by dilution with water, may be applied by spraying, atomizing, dusting, scattering, drenching or treating the seed or mixing with the seed, depending on the selected formulation. The use forms depend on the intended purposes; in any case, they should ensure the finest possible distribution of the active substances according to the invention.

In addition to the active substance compositions according to the invention, the formulations comprise, as a rule, a solid or liquid carrier and adjuvants which are conventionally used in the formulation of plant protection products. Examples of adjuvants which are conventionally used in the formulation of plant protection products are surface-active substances (for example the abovementioned dispersants, protective colloids, emulsifiers, wetters and adhesives) and the above-mentioned organic and inorganic thickeners, bactericides, antifreeze agents, antifoams, if appropriate colorants.

Examples of liquid carriers are mineral oil fractions of medium to high boiling point such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, or water.

Solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders, or other solid carriers.

The examples which follow are intended to illustrate the invention.

I. Assay for Determining the Hydrolase Activity Regarding the Hydrolysis of p-Nitrophenyl Acetate
p-Nitrophenyl acetate is employed in the form of a stock solution in dimethyl sulfoxide/isopropanol (1:1 V/V) at a concentration of 5 mg/ml.
The lipase to be tested was employed as a stock solution in 0.1% BSA (bovine serum albumin) solution at a concentration of 1 mg/ml. For the test, this solution was diluted with 0.1% strength BSA solution to an enzyme concentration of 0.001 mg/ml.
The test was carried out in 1.5 ml Eppendorf vessels. In each case one blank determination and three duplication determinations were carried out.
To carry out the assay, 650 μl of deionized water, 50 μl of lipase in 0.1% BSA (0.001 mg/ml), 100 μl MES buffer (1 M) and 100 μl of the p-nitrophenyl acetate stock solution were placed into the Eppendorf vessels.
The vessels were incubated at the desired temperature in a water bath over a period of 2 minutes. After 2 minutes, the reaction was quenched by addition of 100 μl 1M hydrochloric acid. Thereafter, the amount of nitrophenol formed was determined by HPLC.

The HPLC determination was carried out with the aid of a reverse-phase column (Merck HiBar RT 250-4, Licrosorb RP18 (5 μm). The flow rate was 1.00 ml/minute. The injection volume was 10.0 μl. Detection was accomplished by means of UV spectroscopy at 280 nm and 326 nm. The eluent used was a gradient of 0.1% trifluoroacetic acid in water (eluent A) and 0.1% trifluoroacetic acid in acetonitrile (eluent B).

II Assay for Determining the Hydrolase Activity with Regard to the Polymer P
To this end, 100 mg of polymer P and lipase (1 mg/ml) were shaken at 22° C. and 110 rpm in 50 ml of potassium dihydrogen phosphate buffer (5 mM KH₂PO₄, pH 8). The pH value was determined at regular intervals. A significant change in pH after one day indicates the degradation of the polymer by the lipase.

### III Preparation of the Active Substance Compositions According to the Invention:

Starting Materials:
polycaprolactone: Tone® polymer P767 E from Dow Plastics (number-average molecular weight 50 000 g/mol),
lipase B from Candida antarctica (Structure 1994, 2, p. 293-298)
triticonazole (purity>98%)
lipase from Burkholderia plantarii (Burkholderia glumae), as described in J. prakt. Chem. 1997, 339, p. 381-384 (Swiss-prot No. Q 05489),
polyvinylpyrrolidone: polyvinylpyrrolidone powder with a K value of approximately 17
(Fikentscher K value in water: Kollidon 17 PF from BASF Aktiengesellschaft).

### EXAMPLE 1

### Preparation of an Active Substance Composition According to the Invention by Spray Drying

In a suitable vessel, 10 g of polycaprolactone, 1.8 g of triticonazole, 0.7 g of Candida antarctica lipase and 5 g of the polyvinylpyrrolidone powder were dissolved in 200 g of tetrahydrofuran. The resulting solution was sprayed into a stream of drying gas in a laboratory spray tower from Büchi. The inlet temperature of the drying gas was 140° C. and the outlet temperature 80° C. In this manner, a powder with a primary particle size in the range of from 1 to 100 μm was obtained.

The remaining activity after spray drying was determined with the aid of the activity test described under I. To this end, 1 mg/ml capsules were dispersed in 0.1% strength aqueous BSA solution, and, after a certain period of time had elapsed, the enzyme activity of the suspension was determined using the test described under I. The activity determined was converted into the enzyme content per gram of powder.

### REFERENCE EXAMPLE 2

To check the encapsulation efficacy, various powders (without enzyme) with different polycaprolactone and polyvinylpyrrolidone contents were prepared analogously to example 1. The active substance content was 10% by weight. The powders had a primary particle size in the range of from 1 to 100 μm.

The resulting powder was placed in water and stored for one hour at 22° C. After one hour, the capsules were removed, dissolved in tetrahydrofuran, the active substance content which remained in the capsules was determined by means of UV-VIS and compared with the active substance content of an untreated sample of the powder. The results are shown in the table which follows.

| | PCL [% by | PVP [% by | Active substance |
|---|---|---|---|
| Experiment | weight]¹⁾ | weight]²⁾ | [%]³⁾ |
| A | 90 | 0 | 95 |
| B | 75 | 25 | 70 |
| C | 50 | 40 | 55 |
| D | 30 | 60 | 12 |

¹⁾Polycaprolactone, based on the total weight of the powder
²⁾Polyvinylpyrrolidone, based on the total weight of the powder
³⁾Remaining amount of active substance in the powder, based on 100% active substance in the freshly prepared powder

### EXAMPLE 3

### Determination of the Release of the Active Substance

Since the enzymes have different activities at different temperatures, the compositions according to the invention can be used for releasing an active substance in a temperature-dependent manner. This is demonstrated with reference to the following example.

To this end, an active substance composition with the following composition was prepared analogously to example 1:

66.7% by weight polycaprolactone,
19.3% by weight polyvinylpyrrolidone,
10% by weight triticonazole and
4% by weight Burkholderia plantarii lipase (Burkholderia glumae, Swiss-Prot No. Q 05489).

The resulting powder (primary particle size in the range of from 1 to 100 μm) was suspended in water and stored for 1 hour or 6 hours at 5° C. or 22° C. After the respective period of time had elapsed, the capsules were separated off, dissolved in tetrahydrofuran, and the remaining active substance content was determined by means of UV/VIS measurement and compared with the active substance content of the untreated sample. The results are shown in the table which follows.

Percentage of active
substance¹⁾

| Time | 22° C. | 5° C. |
|---|---|---|
| 1 h | 70% | 95% |
| 6 h | 20% | 85% |

¹⁾Remaining percentage of active substance in the powder based on 100% active substance in the reference sample.

### EXAMPLE 4

### Use Example

To confirm that the presence of the enzyme affects the release, the stunting, triggered by triticonazole, and the effect on the germination rate were studied on soya kernels.

To this end, the following two pulverulent active substance compositions with the following composition were prepared analogously to Example 1:

### Active Substance Composition 4.1

29% by weight polycaprolactone,
58% by weight polyvinylpyrrolidone,
10% by weight triticonazole and
3% by weight Burkholderia plantarii lipase (Burkholderia glumae, Swiss-Prot No. Q 05489).

### Active Substance Composition 4.2 (not According to the Invention)

30% by weight polycaprolactone,
60% by weight polyvinylpyrrolidone and
10% by weight by weight triticonazole.

The powders had a primary particle size in the range of from 0.1 to 10 μm.

Soya kernels cv. Lory were treated with a commercially available FS formulation of triticonazole and with two aqueous dispersions of the active substance composition 4.1 and 4.2, respectively. To this end, in each case 50 soya kernels were treated with the samples at application rates of 10 and 20 g triticonazole per 100 kg of seed, respectively, and sown into polystyrene dishes filled with sand. The seedlings were grown in the greenhouse at temperatures of between 18 and 22° C. and 12 hours' light. 27 days after sowing, the germination rate and the average plant height were determined for each seed box.

Result of the application example

| | | | Germination | |
|---|---|---|---|---|
| | | | rate | Plant height |
| | | Application rate | 27 DAT¹⁾ | 27 DAT¹⁾ |
| # | Treatment | g a.i./100 kg | % | cm |
| 1 | Untreated control | — | 72 | 47.5 |
| 2 | Active substance | 10 | 84 | 14.5 |
| | composition 4.2 | | | |
| 3 | Active substance | 20 | 88 | 8.0 |
| | composition 4.2 | | | |
| 4 | Active substance | 10 | 86 | 12.0 |
| | composition 4.2 | | | |
| 5 | Active substance | 20 | 80 | 5.0 |
| | composition 4.2 | | | |
| 6 | FS formulation | 10 | 84 | 9.0 |
| 7 | FS formulation | 20 | 78 | 4.5 |

¹⁾27 DAT: Measurement was carried out 27 days after sowing

It can be seen from the data in Table 1 that the stunting is greater in plants which have been treated with enzyme-comprising composition than in those without enzyme. This shows that the active substance is released more rapidly from the capsule as the result of the enzyme. At the same time, it can be seen that, due to the encapsulation, the germination rate and the plant height are affected to a lesser extent than in the conventional FS formulation.

1-20. (canceled)

21. An active substance composition in the form of finely divided, active substance-containing particles, comprising:
a) at least one plant protectant;
b) at least one polymer P which is not soluble in water and which is degradable by enzymatic hydrolysis, in an amount of at least 20% by weight based on the total amount of the components of the active substance-containing particles, and
c) at least one hydrolase EC 3.1) of the EC classes
EC 3.1.1 (carboxylic ester hydrolases),
EC 3.1.2 (thioester hydrolases),
EC 3.1.3 (phosphoric acid monoester hydrolases),
EC 3.1.4 (phosphoric acid diester hydrolases),
EC 3.1.5 (triphosphoric acid monoester hydrolases),
EC 3.1.6 (sulfuric ester hydrolases),
EC 3.1.7 (diphosphoric monoester hydrolases),
EC 3.1.8 (phosphoric triester hydrolases);
glycosylases (EC 3.2), for example glycosylases from the EC classes
EC 3.2.1 (glycosidases, enzymes which hydrolyze O- and S-glycosyl compounds),
EC 3.2.2 (enzymes which hydrolyze N-glycosyl compounds),
EC 3.2.3 (enzymes which hydrolyze S-glycosyl compounds);
enzymes which hydrolyze ether bonds (EC 3.3), from the EC classes
EC 3.3.1 (thioether and trialkylsulfonium hydrolases)
EC 3.3.2 (ether hydrolases)
enzymes which hydrolyze peptide bonds, peptidases (EC 3.4), from the EC classes
EC 3.4.11 (aminopeptidases)
EC 3.4.13 (dipeptidases)
EC 3.4.14 (dipeptidylpeptidases and tripeptidylpeptidases)
EC 3.4.15 (peptidyldipeptidases)
EC 3.4.16 (serine type carboxypeptidases)
EC 3.4.17 (metallocarboxypeptidases)
EC 3.4.18 (cysteine-type carboxypeptidases)
EC 3.4.19 (omega peptidases)
EC 3.4.21 (serine endopeptidases)
EC 3.4.22 (cysteine endopeptidases)
EC 3.4.23 (aspartic endopeptidases)
EC 3.4.24 (metalloendopeptidases)
EC 3.4.25 (threonine endopeptidases)
EC 3.4.99 (endopeptidases with unknown catalytic mechanism)
enzymes which hydrolyze carbon-nitrogen bonds which are not amide bonds (EC 3.5), from the EC classes
EC 3.5.1 (enzymes which catalyze the hydrolysis of linear amides)
EC 3.5.2 (enzymes which catalyze the hydrolysis of cyclic amides)
EC 3.5.3 (enzymes which catalyze the hydrolysis of linear amidines)
EC 3.5.4 (enzymes which catalyze the hydrolysis of cyclic amidines)
EC 3.5.99 (enzymes which catalyze the hydrolysis of other compounds)
enzymes which hydrolyze acid anhydrides (EC3.6) of the EC classes
EC 3.6.1 (enzymes which catalyze the hydrolysis of phosphorus-comprising anhydrides)
EC 3.6.2 (enzymes which catalyze the hydrolysis of sulfonyl-comprising anhydrides)
EC 3.6.3 (enzymes which are catalytically active on acid anhydrides)
EC 3.6.4 (enzymes which are catalytically active on acid anhydrides)
EC 3.6.5 (enzymes which are catalytically active on GPT)
enzymes which hydrolyze carbon-carbon bonds (EC 3.7),
EC 3.7.1 (enzymes which catalyze the hydrolysis of ketone-containing substrates)
enzymes which hydrolyze halogen-carbon bonds (EC 3.8), from the EC class EC 3.8.1 (enzymes which hydrolyze C-halogen compounds)
enzymes which hydrolyze phosphorus-nitrogen bonds (EC3.9)
enzymes which hydrolyze sulfur-nitrogen bonds (EC3.10)
enzymes which hydrolyze carbon-phosphorus bonds (EC3.11)
enzymes which hydrolyze sulfur-sulfur bonds (EC3.12) and
enzymes which hydrolyze carbon-sulfur bonds (EC3.13)
wherein at least 90% by weight of the active substance-containing particles of the active substance composition do not exceed a diameter of 500 μm (D₉₀-value) and where components a), b) and c) account for at least 30% by weight of the active substance particles.

22. The active substance composition of claim 21, wherein the polymer P has a multiplicity of ester groups in the polymer backbone and the enzyme is selected from
EC 3.1 of the EC classes
EC 3.1.1 (carboxylic ester hydrolases),
EC 3.1.2 (thioester hydrolases),
EC 3.1.3 (phosphoric acid monoester hydrolases),
EC 3.1.4 (phosphoric acid diester hydrolases),
EC 3.1.5 (triphosphoric acid monoester hydrolases),
EC 3.1.6 (sulfuric ester hydrolases),
EC 3.1.7 (diphosphoric monoester hydrolases), and
EC 3.1.8 (phosphoric triester hydrolases).

23. The active substance composition of claim 22, wherein the enzyme is a carboxylic ester hydrolase selected from a lipase from Aspergillus oryzae, a cutinase from Fusarium solani, a lipase from Burkholderia plantarii and a lipase from Candida antarctii.

24. The active substance composition of claim 22, wherein the polymer P is selected among polylactides, polycaprolactone, block copolymers of polylactide and polyethylene glycol, and block copolymers of polycaprolactone and polyethylene glycol.

25. The active substance composition of claim 22, wherein the at least one hydrolase is selected from the group of the carboxylic ester hydrolases (EC 3.1.1).

26. The active substance composition of claim 25, wherein said hydrolase is selected from the group of the lipases (EC 3.1.1.3) and the group of cutimates (EC 3.1.1.74).

27. The active substance composition of claim 21, wherein the at least one hydrolase is essentially inactive at a temperature of below 10° C.

28. The active substance composition of claim 21, wherein the polymer P has a glass transition temperature or a melting point above 40° C.

29. The active substance composition of claim 21, wherein the polymer P in the active substance-containing particles is present in an amount of from 20 to 95% by weight based on the total amount of the components of the active substance-containing particles.

30. The active substance composition of claim 21, wherein the at least one plant protectant is selected among organic compounds which have a fungicidal, insecticidal, acaricidal and/or nematicidal activity.

31. The active substance composition of claim 21, wherein the plant protectant in the active substance-containing particles is present in an amount of 5 to 30% by weight, based on the total amount of the components of the active substance particles.

32. The active substance composition of claim 21, comprising up to 70% by weight of a polymer which is soluble in water at 20° C.

33. The active substance composition of claim 21, wherein the water-soluble polymer is selected from the group consisting of polyvinylpyrrolidones, vinylpyrrolidone/vinyl acetate copolymers, polyvinylformamides, partially hydrolyzed polyvinylformamides, homo- and copolymers of acrylic acid, homo- and copolymers of methacrylic acid, homo- and copolymers of acrylamide, polyethyleneimines, polyvinylamines, polycaprolactams, polyvinyl alcohols, partially hydrolyzed polyvinyl acetates with a degree of hydrolysis of >50%, cellulose, cellulose derivatives, modified starches and starch derivatives, dextrans and poly-C₂-C₃-oxyalkylenes.

34. A process for the preparation of an active substance composition according to claim 21, comprising the mixing of the components of the active substance-containing particles and the further processing of the mixture to give a finely divided powder in which at least 90% by weight of the active-substance-comprising particles of the active substance composition do not exceed a diameter of 500 μm (D₉₀ value).

35. The process of claim 34, wherein the preparation of the active substance composition further comprises the dissolving or dispersing of the components of the active substance-containing particles in a suitable solvent or diluent and subsequent spray-drying of the resulting dispersion or solution.

36. Seed comprising an active substance composition of claim 21.

37. An aqueous composition comprising an active substance composition of claim 21 in the form of finely divided active substance-containing particles which are dispersed in an aqueous medium.

38. A method of preparing an active substance-containing seed of claim 36, comprising contacting a conventional seed with said active substance composition.
